(19) United States
(12) Patent Application Publication
(10) Pub. No.: US 2018/0328943 A1
(43) Pub. Date: Nov. 15, 2018

(54) IMMUNOMODULATORY COMPOSITIONS AND METHODS OF USE THEREOF

(71) Applicant: Enzo Biochem, Inc., New York, NY (US)

(72) Inventors: Ante Tocilj, Syosset, NY (US); Jack Coleman, Easty Northport, NY (US); Elazar Rabbani, New York, NY (US); James J. Donegan, Long Beach, NY (US)

(73) Assignee: Enzo Biochem, Inc., New York, NY (US)

(21) Appl. No.: 16/039,620

(22) Filed: Jul. 19, 2018

Related U.S. Application Data
(63) Continuation-in-part of application No. 15/719,821, filed on Sep. 29, 2017.
(60) Provisional application No. 62/535,047, filed on Jul. 20, 2017.
(60) Provisional application No. 62/402,248, filed on Sep. 30, 2016.

(51) Int. Cl. G01N 33/68

(52) U.S. Cl. CPC G01N 33/6866; G01N 2333/57

(57) ABSTRACT

Provided are immunomodulatory pharmaceutical and non-pharmaceutical compositions that include alpha-synuclein and at least one preselected antigen, such as at least one preselected peptide antigen or immunogen. Also provided are methods for modulating immune activity toward at least one preselected antigen in an at least substantially antigen-specific manner that include administering such a composition to a human patient or to a non-human mammalian subject. Still further provided are enhanced assay methods for quantifying antigen-specific cellular responses, such as cytokine release, to preselected antigens.

### CROSS-REFERENCE TO RELATED APPLICATIONS

This application is a continuation in part of U.S. application Ser. No. 15/719,821 filed Sep. 29, 2017, which claims the benefit of U.S. provisional application Ser. No. 62/535,047 filed Jul. 20, 2017 and 62/402,248 filed Sep. 30, 2016, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 16, 2018, is named ENZ-112-CIP_SL_ST25.txt and is 45,539 bytes in size.

### FIELD OF THE INVENTION

The invention relates to the field of antigen-specific immune modulation.

### BACKGROUND

The immune system and its regulation are central to our well-being. A healthy immune system recognizes and eliminates pathogens, pre-cancerous cells and other “non-self” entities, while maintaining a state of non-reactiveness toward normal self cells and tissues. When this state of non-reactiveness to self-antigens breaks down, autoimmune disease may result. Indeed, many chronic inflammatory and tissue-destructive diseases are autoimmune diseases, including, for example, age-related macular degeneration (AMD), uveitis, Crohn's disease, rheumatoid arthritis, systemic lupus erythematosus, and multiple sclerosis. Over eighty autoimmune diseases are known.

What is needed and provided by the present invention are new compositions and methods for modulating immune activity, i.e., for promoting immune reactivity or immune suppressiveness, with respect to preselected antigens.

### SUMMARY OF THE INVENTION

One embodiment of the invention provides an immunomodulatory composition, such as an immunomodulatory pharmaceutical composition, including a mixture of:

(i) a first component including
(a) at least partially purified HLA protein or fragments thereof, such as mammalian, for example human,
(b) whole blood, such as mammalian, for example human, or a cellular fraction thereof, such as a density gradient fraction thereof, such as but not limited to a white blood cell and/or red blood cell (erythrocytes) and/or platelet fraction/layer thereof, or a cell membrane fraction/preparation of any of the foregoing or an extract of any of the foregoing, such as a protein extract, a lipid extract, a carbohydrate extract, a small molecule extract or any combination thereof, and/or
(c) alpha-synuclein protein, such as mammalian alpha-synuclein protein, such as human alpha-synuclein protein, or fragments thereof; and

(ii) at least one preselected antigen or immunogen, such as at least one preselected peptide antigen, at least one preselected protein antigen, at least one preselected carbohydrate antigen, at least one preselected lipid antigen, and/or at least one preselected glycolipid antigen.

At least the first component or only the first component may be heat-treated, for example, heat-treated at or above 100° C. for at least 1 or 2 or 3 or 4 or 5 minutes. Heat-treatment may include or consist of autoclaving. The at least partially purified HLA protein or other proteins may be at least substantially denatured. The proteins may at least partially, such as at least substantially, be fragmented into peptides.

For any of the embodiments throughout this disclosure, the antigen or immunogen may be a molecule that is not an HLA molecule and/or is not alpha-synuclein (and/or is not a sequence fragment of either).

The composition may, for example, be a liquid composition or an at least substantially dry composition, such as a powder. Dry forms may be prepared by drying a liquid mixture of the components, for example, by lyophilization or any method known in the art. The composition may include one or more pharmaceutically acceptable excipients.

A related embodiment provides a method for manufacturing an immunomodulatory composition, such as an immunomodulatory pharmaceutical composition, including the steps of:

providing a first component including
(a) at least partially purified HLA protein or fragments thereof, such as mammalian, for example human,
(b) whole blood, such as mammalian, for example human, or a cellular fraction thereof, such as a density gradient fraction thereof, such as but not limited to a white blood cell and/or red blood cell (erythrocytes) and/or platelet fraction/layer thereof, or a cell membrane fraction/preparation of any of the foregoing or an extract of any of the foregoing, such as a protein extract, a lipid extract, a carbohydrate extract, a small molecule extract or any combination thereof, and/or
(c) alpha-synuclein protein, such as mammalian alpha-synuclein protein, such as human alpha-synuclein protein, or fragments thereof; and

providing a second component including at least one preselected antigen or immunogen, such as at least one preselected peptide antigen, at least one preselected protein antigen, at least one preselected carbohydrate antigen, at least one preselected lipid antigen, and/or at least one preselected glycolipid antigen; and

mixing the first component and the second component, for example, under aqueous conditions.

The method may further include heat-treating at least the first component, such as only the first component prior to the mixing step. The method may include mixing the two components and then heat-treating the mixture. The method may include separately heat-treating the first and second components prior to the mixing step.

The method may further include at least substantially drying the liquid mixture to obtain an at least substantially dry form, such as a powder, by, for example, lyophilizing the liquid mixture or otherwise drying it. One or more excipients may be admixed before and/or after the drying step.

The antigen (or immunogen) may, for example, be a molecule that is not an HLA molecule and/or is not alpha-synuclein. For example, the antigen may be a peptide, such as a synthetic peptide, that is not a sequence fragment of an HLA molecule or alpha-synuclein.

A further embodiment of the invention provides a method for modulating the immune response in a mammal to at least one preselected antigen that includes administering to the mammal an immunomodulatory pharmaceutical composition as described within. Said administration may be parenteral or non-parenteral. The antigen-specific modulation of the immune response may be immunostimulatory or immunosuppressive (tolerogenic).

A further embodiment of the invention provides a method for modulating the immune response in a mammal, such as a human, to at least one preselected antigen that includes:

coadministering to the mammal:
(i) one or more of
   (a) at least partially purified HLA protein or fragments thereof, such as mammalian, for example human,
   (b) whole blood, such as mammalian, for example human, or a cellular fraction thereof, such as a density gradient fraction thereof, such as but not limited to a white blood cell and/or red blood cell (erythrocytes) and/or platelet fraction/layer thereof, or a cell membrane fraction/preparation of any of the foregoing or an extract of any of the foregoing, such as a protein extract, a lipid extract, a carbohydrate extract, a small molecule extract or any combination thereof, and
   (c) alpha-synuclein protein, such as mammalian alpha-synuclein protein, such as human alpha-synuclein protein, or fragments thereof; and

(ii) at least one preselected antigen or immunogen, such as at least one preselected peptide antigen, at least one preselected protein antigen, at least one preselected carbohydrate antigen, at least one preselected lipid antigen, and/or at least one preselected glycolipid antigen.

Any one or more of the compositions under (i) may be treated, such as heat-treated, in any of the manners described herein.

Still another embodiment of the invention provides an assay method for determining whether cells in a sample of cells mount an antigen-specific response to one or more preselected antigens and/or for quantifying the extent to which cells in a sample of cells mount an antigen-specific response to one or more preselected antigens, said method embodiment including the steps of: providing an isolated sample of cells, such as a sample of blood cells, such as whole blood, or a white blood cell fraction or PBMCs or T-cells; providing alpha-synuclein protein and/or a fragment thereof; providing at least one, such as one, preselected antigen; contacting the sample of cells with both the alpha-synuclein and/or fragments thereof and the at least one preselected antigen; and measuring the resulting cellular response, such as the release of a cytokine, to said contacting.

Other objects and advantages of the invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE DRAWINGS

FIGS. 1A and 1B show the effects of a null control treatment on healthy PBMCs (FIG. 1A) and the effect of heat-treated whole blood on PBMCS (FIG. 1A), respectively, both with no added antigens/epitopes, with the top panels showing the cell cycle distribution of treated cells and the bottom panels showing the expression of interferon-gamma (IFNg) in treated cells.

FIG. 2A shows the cell cycle distribution of cells in healthy PBMCs (control case). FIG. 2B shows the cell cycle distribution of PBMCs treated with whole blood (not heat-treated). FIG. 2C shows the cell cycle distribution of PBMCs treated with serum. FIG. 2D shows the cell cycle distribution of PBMCs treated with a heat-treated red blood cell (RBC) density gradient fraction (referred to as the “immune enhancer fraction;” “RBC;” and “IE” herein).

FIGS. 3A and 3B show cell cycle distribution of PBMCs treated with control and various heat-treated blood fractions for allogeneic blood (FIG. 3A) and autologous blood (FIG. 3B) indicating that that the apoptosis-inducing activity of whole blood is predominantly present in the RBC fraction, and is independent of the donor source.

FIGS. 4A-C show the effect of control (FIG. 4A), recombinant HLA-B protein (rHLA-B; FIG. 4B), and recombinant HLA-G protein (rHLA-G; FIG. 4C) on the cell cycle distribution of PBMCs, indicating apoptosis-inducing activity of the rHLA-G and rHLA-B proteins on the PBMCs.

FIGS. 5A and 5B show the effect of various treatment on the percent of PBMCs in the sub-G1 phase (FIG. 5A) and the S+G2 phase (FIG. 5B).

FIGS. 6A and 6B show the effect of various blocking factors on the ability of the immune enhancer (heat-treated RBC fraction from density gradient separation) to affect the percentage of PBMCs in the sub-G1 phase (FIG. 6A; indicative of apoptosis induction) and the S+G2 phase (FIG. 6B; indicative of proliferation).

FIG. 7 shows the effect of different doses of IE on the ability of phytohemagglutinin (PHA) to induce interferon-gamma (IFNγ) expression in PBMCs, as measured by mRNA detection.

FIGS. 8A-8C show the effects on cell cycle distribution of control (FIG. 8A), Hepatitis B virus S-antigen (“HBV-SA;” FIG. 8B) alone, and HBV-A plus IE (FIG. 8C) on PBMCs from healthy human subjects (top panels) and HBV patients (bottom panels).

FIGS. 9A-D show the effect of control (FIG. 9A), HBV-SA alone (FIG. 9B), IE alone (FIG. 9C), and HBV-SA plus IE (FIG. 9D) on the expression of the Foxp3 immune suppression marker in PBMCs from healthy human subjects (top panels) and HBV patients (bottom panels).

FIG. 10 shows the percent increase in Foxp3 mRNA for control and different treatments in the experiment shown in FIGS. 9A-D.

FIGS. 11A and 11B show the effect of HBV-SA plus IE (FIG. 11A) and HBV-SA plus IE plus anti-HLA class I antibody (aHLA) on the expression of the Foxp3 immune suppression marker in PBMCs from healthy human subjects (top panels) and HBV patients (bottom panels).

FIGS. 12A-D show the effect of control (FIG. 12A), HBV-SA alone (FIG. 12B), IE alone (FIG. 12C), and HBV-SA plus IE (FIG. 12D) on the expression of interferon-gamma in PBMCs from healthy human subjects (top panels) and HBV patients (bottom panels).

FIGS. 13A and 13B show the effect of HBV-SA plus IE (FIG. 13A) and HBV-SA plus IE plus anti-HLA class I antibody (aHLA) on the expression of interferon-gamma (IFNg) in PBMCs from healthy human subjects (top panels) and HBV patients (bottom panels).

FIG. 14 shows the antigen-dependent, immune stimulation-enhancing activity of human alpha-synuclein protein (as measured by IFN-gamma release) during antigen challenge of whole blood from a subject recently immunized with the subject antigen.

### DETAILED DESCRIPTION

One aspect of the invention is based on the inventors' discovery that heat-treated blood (HTB) can modulate immune responses to antigens. When investigating the combination of HTB and HBsAg with PBMCs from HBV infected patients, the response to HBsAg alone was limited, but when HTB was added, a much stronger response was seen. The effect is antigen specific since HTB by itself gave no response. This result remains unchanged whether the blood is from an autologous or heterologous source, or even from the pooled blood of multiple donors, meaning HTB used to treat patients could be either from the patient themselves which they could donate before the study begins, or as an off-the-shelf reagent from allogeneic donors.

Through ficoll density gradient separation, it was found that the active factor or factors are present in at least the red blood cell (RBC) layer of blood. Soluble (recombinant) HLA was shown to have a similar effect. In in vitro experiments on PBMCs, heat-treated soluble HLA gave similar results as both heat-treated whole blood and the heat-treated RBC density gradient fraction. The effect with the HLA was not as strong as with the heat-treated whole blood or that with the heat-treated RBC fraction, possibly indicating other contributory factors or the presence of a concentration effect. It was further discovered that the heat treatment increases the effectiveness of the soluble HLA as an immune enhancer.

Heat treatment may, for example, be performed at a temperature of at least 95° C., such as at least 100° C., such as at least 110° C., such as at least 120° C. for at least 1 minute, such as but not limited to 1 minute, at least 15 minutes or 15 minutes, at least 20 minutes or 20 minutes, at least 25 minutes or 25 minutes, or at least 30 minutes or 30 minutes. Heat treatment may, for example be conducted at a temperature in the range of 100-130° C. Heat treatment may include or consist of autoclaving, for example, for 1 minute to three hours, such as 1-30 minutes, such as 5-25 minutes, such as 10-20 minutes, or any subrange or number of minutes within said ranges.

The dose, such as daily dose, of heat-treated blood or blood fraction may, for example, be in the range of 0.5 mg to 5 grams or any amount or subrange of amounts therein, such as 0.5 to 100 mg or 1.0 to 50 mg. Heat treatment may, for example, be performed by autoclaving the material. The dose of a protein or protein extract of the blood or blood fraction or purified or recombinant HLA protein (or fragment(s) thereof) or purified or recombinant alpha-synuclein protein (or fragment(s) thereof) may, for example, be in the range of 100 micrograms to 100 mg, such as 20 mg to 100 mg or any subrange or value therein such as 0.5 to 5 mg. Doses of compositions including such extracts, proteins or fragments thereof and one or more preselected antigens such as protein or peptide antigens may, for example, be in the same weight ranges. Compositions including such a combination of components may, for example, include them in molar ratios of 1:100 to 100:1 of immune enhancer:preselected antigen or any subrange or molar ratio value therein such as 5:1 to 1:5.

Dosing may, for example, be performed thrice daily, twice daily, once daily, every other day, every three days, biweekly or weekly.

Various aspects of the invention are further illustrated by the appended drawings and experimental results shown therein. Blood products indicated were made from human whole blood or blood fractions. Blood fractions were isolated by ficol gradient centrifugation using Histopaque (Sigma) and collecting either the RBC or serum fractions as indicated in each experiment. The whole blood or fraction was then autoclaved for 20 minutes, then resuspended to twice their original volume using PBS and sonicated for 30 minutes to restore solubility. PBMCs which had been frozen in liquid nitrogen were thawed, washed in RPMI 1640, then resuspended in RPMI 1640 complete medium, treated as indicated, and incubated for between 16 and 72 hours for use, depending on the experiment. Cells were then collected and stained as indicated, and run in a FACS Calibur flow cytometer.

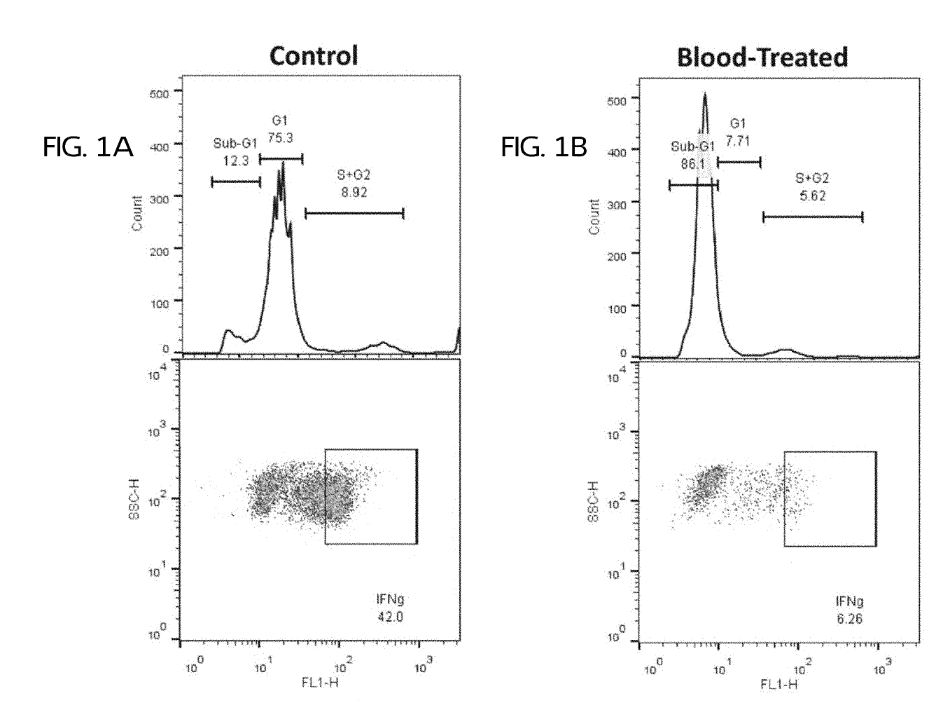

FIGS. 1A and 1B show the effects of a null control treatment on healthy PBMCS (FIG. 1A) and the effect of heat-treated whole blood on PBMCS (FIG. 1A), respectively, both with no added antigens/epitopes, with the top panels showing the cell cycle distribution of treated cells and the bottom panels showing the expression of interferon-gamma (IFNg) in treated cells. The experiment shows that heat-treated whole blood induced apoptosis in the PBMCs and reduced the number of cells expressing the proinflammatory cytokine interferon-gamma.

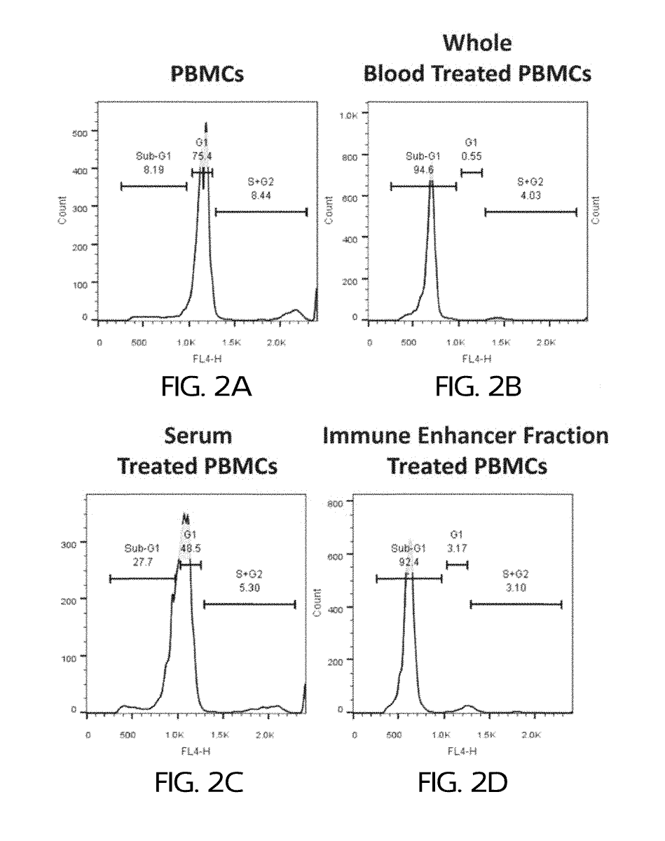

FIG. 2A shows the cell cycle distribution of cells in healthy PBMCs (control case). FIG. 2B shows the cell cycle distribution of PBMCs treated with heat treated whole blood. FIG. 2C shows the cell cycle distribution of PBMCs treated with heat treated serum. FIG. 2D shows the cell cycle distribution of PBMCs treated with a heat-treated red blood cell fraction (referred to as the “immune enhancer fraction;” “RBC;” and “IE” herein).

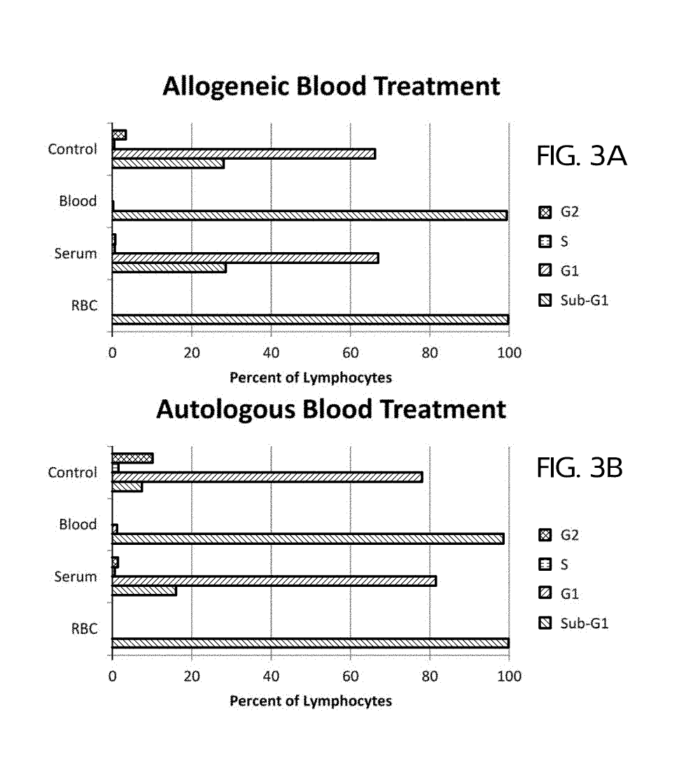

FIGS. 3A and 3B show cell cycle distribution of PBMCs treated with control and various heat-treated blood fractions for allogeneic blood (FIG. 3A) and autologous blood (FIG. 3B) indicating that that the apoptosis-inducing activity of whole blood is predominantly present in the RBC fraction and independent of donor source. Heat-treated IE showed essentially the same extent of apoptosis induction as heat-treated whole blood. The heat-treated serum fraction decreased cell proliferation but did not increase apoptosis. The IE dose was 50 μL.

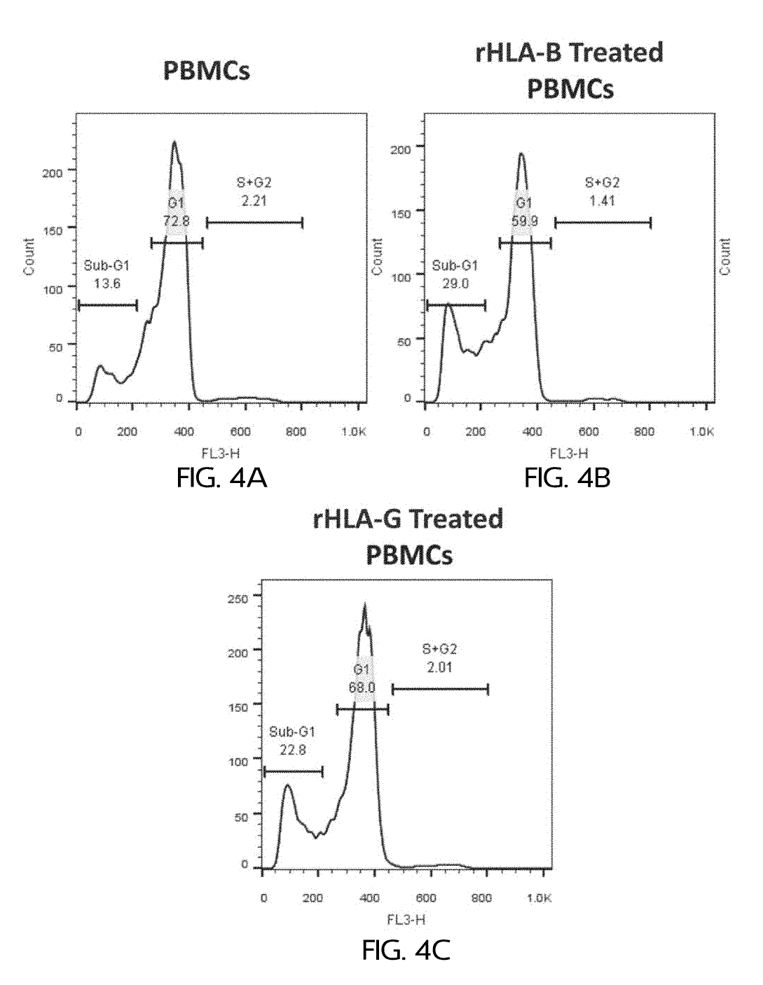

FIGS. 4A-C show the effect of control (FIG. 4A), recombinant HLA-B protein (rHLA-B; FIG. 4B), and recombinant HLA-G protein (rHLA-G; FIG. 4C) on the cell cycle distribution of PBMCs, indicating apoptosis-inducing activity of the rHLA-G and rHLA-B proteins on the PBMCs.

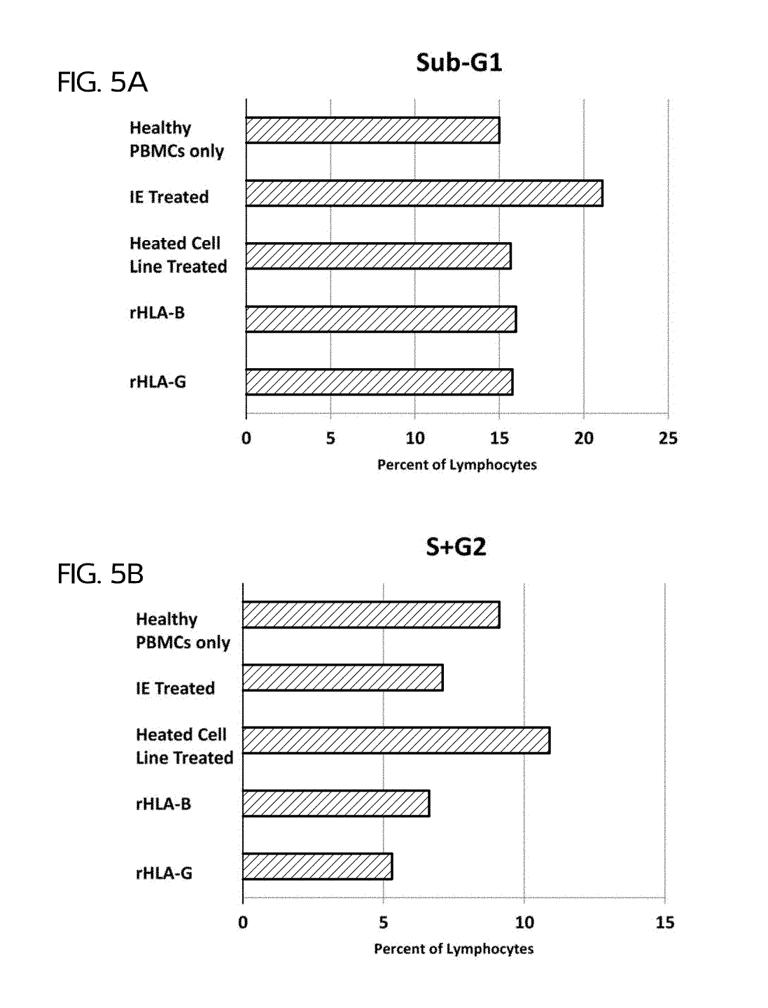

FIGS. 5A and 5B show the effect of various treatment on the percent of PBMCs in the sub-G1 phase (FIG. 5A) and the S+G2 phase (FIG. 5B). The heat-treated cell-line was a HeLa cell line. IE increased apoptosis and decreased proliferation of the lymphocytes (PBMCs). Recombinant HLA proteins (65 ng/mL) gave only minor induction of apoptosis, but decreased proliferation. The heat-treated HeLa cells did not mimic apoptosis induction and, in fact, induced higher proliferation. The IE dose was 5 μL. The lower dose induced less apoptosis compared to the 50 μL dose.

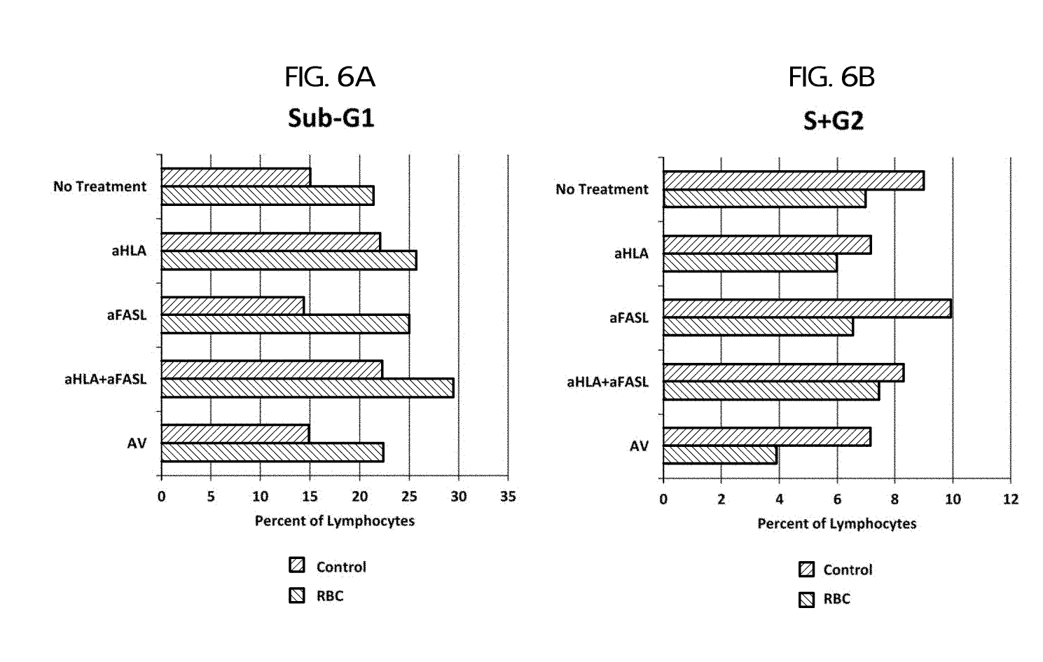

FIGS. 6A and 6B show the effect of various blocking factors on the ability of the immune enhancer (heat-treated RBC density gradient fraction) to affect the percentage of PBMCs in the sub-G1 phase (FIG. 6A) and the S+G2 phase (FIG. 6B). Anti-HLA-I and FasL blocking antibodies do not reverse IE-induced apoptosis. Anti-HLA-I antibody itself induces an increase in apoptosis independent of IE. Annexin V (which blocks phosphatidyl serine) shows no effect on apoptosis, but reduces proliferation in both control and IE treated groups.

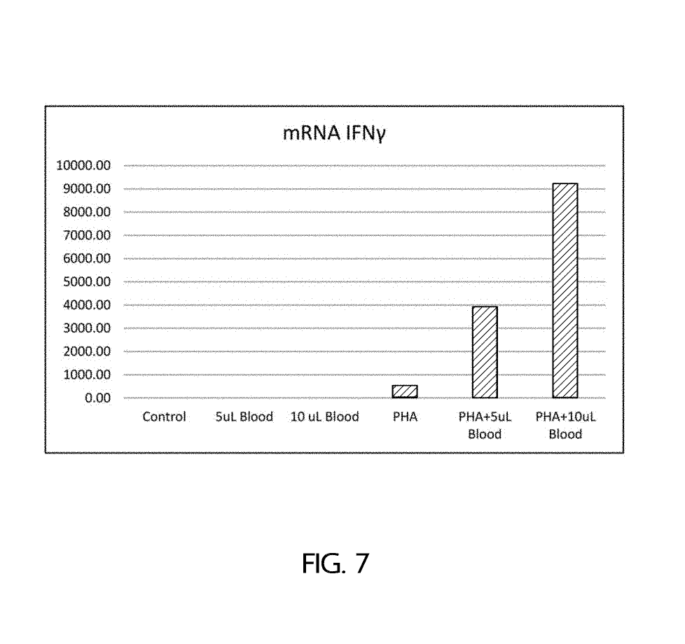

FIG. 7 shows the effect of different doses of IE on the ability of phytohemagglutinin (PHA) to induce interferon-gamma (IFNγ) expression in PBMCs, as measured by mRNA detection. IE stimulated the immune response to PHA in a dose-dependent manner.

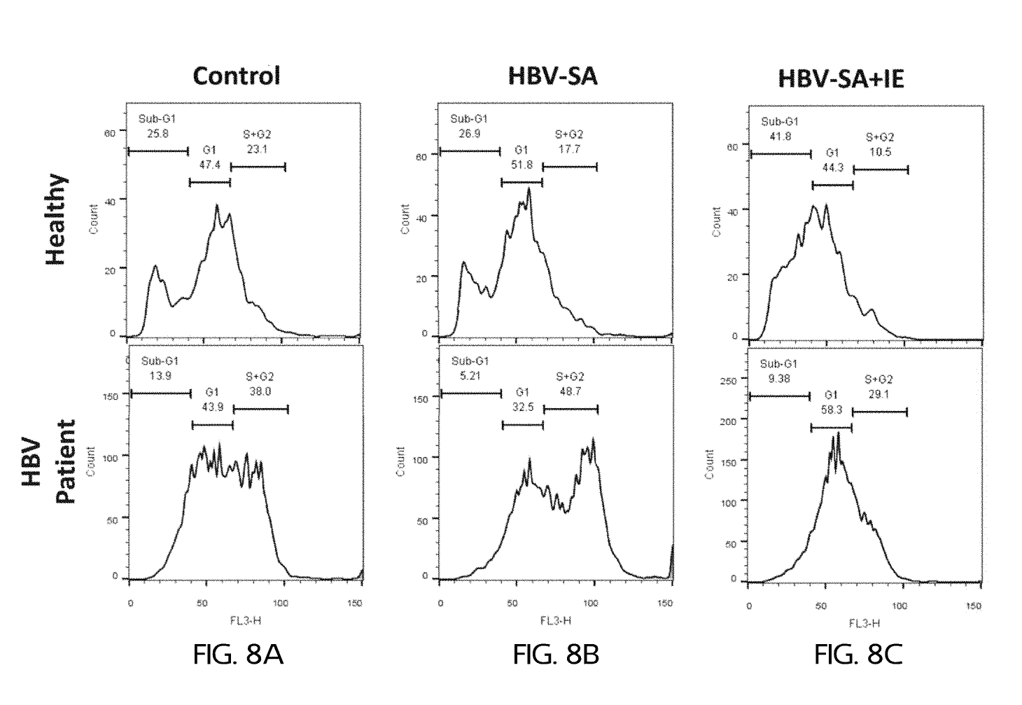

FIGS. 8A-8C show the effects on cell cycle distribution of control (FIG. 8A), hepatitis B virus S-antigen (“HBV-SA;” FIG. 8B) alone, and HBV-A plus IE (FIG. 8C) on PBMCs from healthy human subjects (top panels) and HBV patients (bottom panels). For the healthy control PBMCs (non-HBV-infected subject), the addition of HBV-SA alone had little/no effect while the addition of HBV-SA plus IE had a pronounced pro-apoptotic effect. For PBMCs from HBV-infected subjects, the addition of HBV-SA only shifted the cells toward proliferation (versus control) while the addition of HBV-SA plus IE reduced proliferation versus both control and HBV-SA alone.

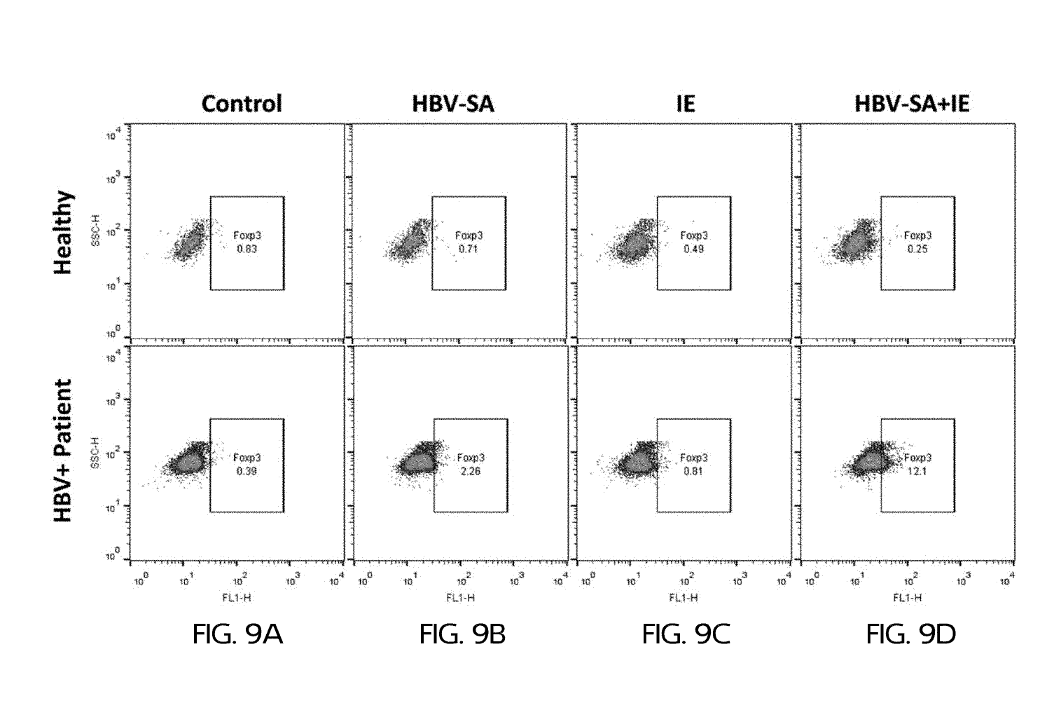

FIGS. 9A-D show the effect of control (FIG. 9A), HBV-SA alone (FIG. 9B), IE alone (FIG. 9C), and HBV-SA plus IE (FIG. 9D) on the expression of the Foxp3 immune suppression (Treg) marker in PBMCs from healthy human subjects (top panels) and HBV patients (bottom panels).

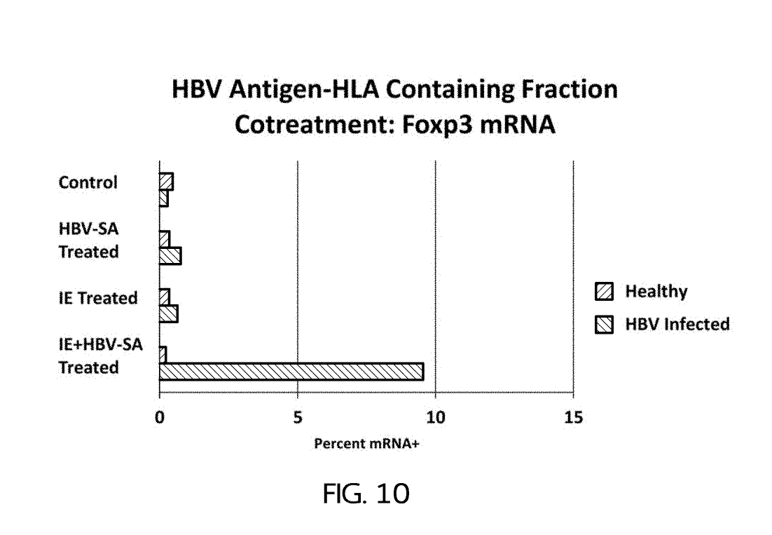

FIG. 10 shows the percent increase in Foxp3 mRNA for control and different treatments in the experiment shown in FIGS. 9A-D. Low-dose IE (5 μL) increased expression of anti-inflammatory marker Foxp3 by cells from an antigen responsive patient in the presence of the antigen. IE alone did not increase expression of Foxp3. Foxp3 expression correlated with antigen-specific suppression in the presence of low-dose IE, in contrast to the general suppression of apoptosis seen with high-dose IE.

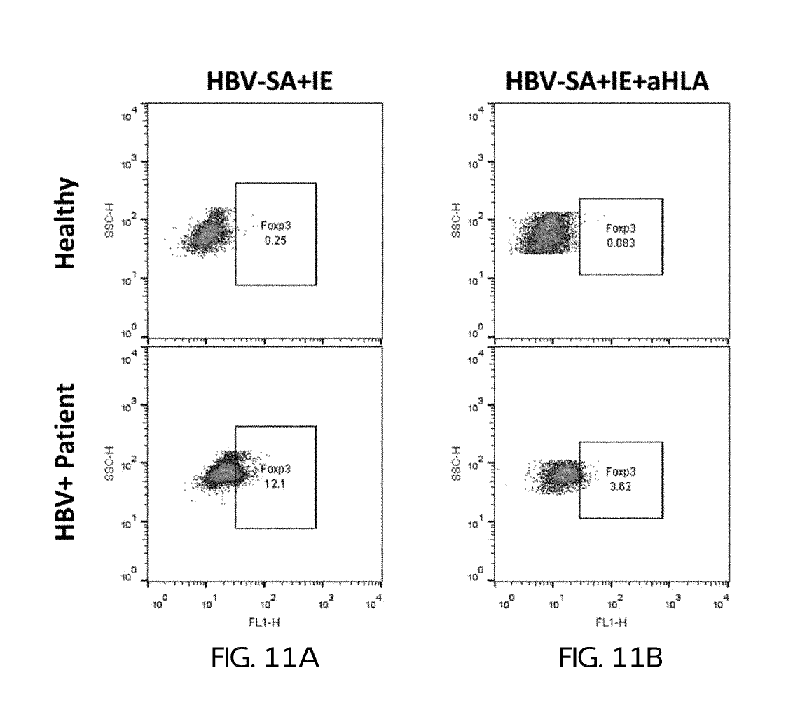

FIGS. 11A and 11B show the effect of HBV-SA plus IE (FIG. 11A) and HBV-SA plus IE plus anti-HLA class I antibody (aHLA) on the expression of the Foxp3 immune suppression marker in PBMCs from healthy human subjects (top panels) and HBV patients (bottom panels). The experiment shows that an anti-HLA class I antibody partially blocks the induction of the Foxp3 marker by HBV-SA plus IE in both healthy PMBCs and PBMCs from HBV-infected subjects.

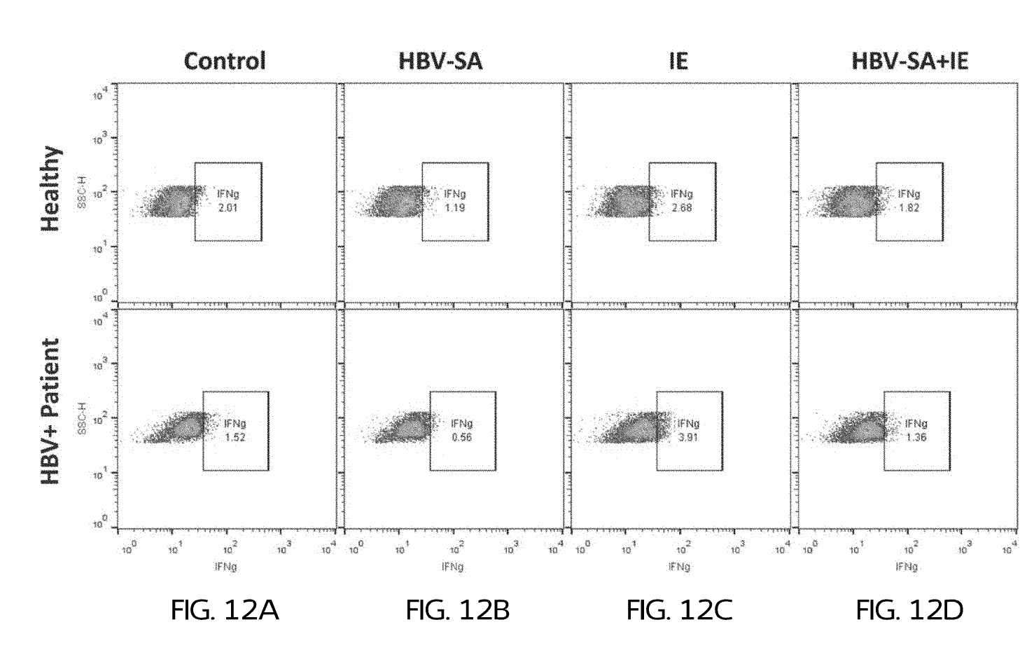

FIGS. 12A-D show the effect of control (FIG. 12A), HBV-SA alone (FIG. 12B), IE alone (FIG. 12C), and HBV-SA plus IE (FIG. 12D) on the expression of interferon-gamma in PBMCs from healthy human subjects (top panels) and HBV patients (bottom panels).

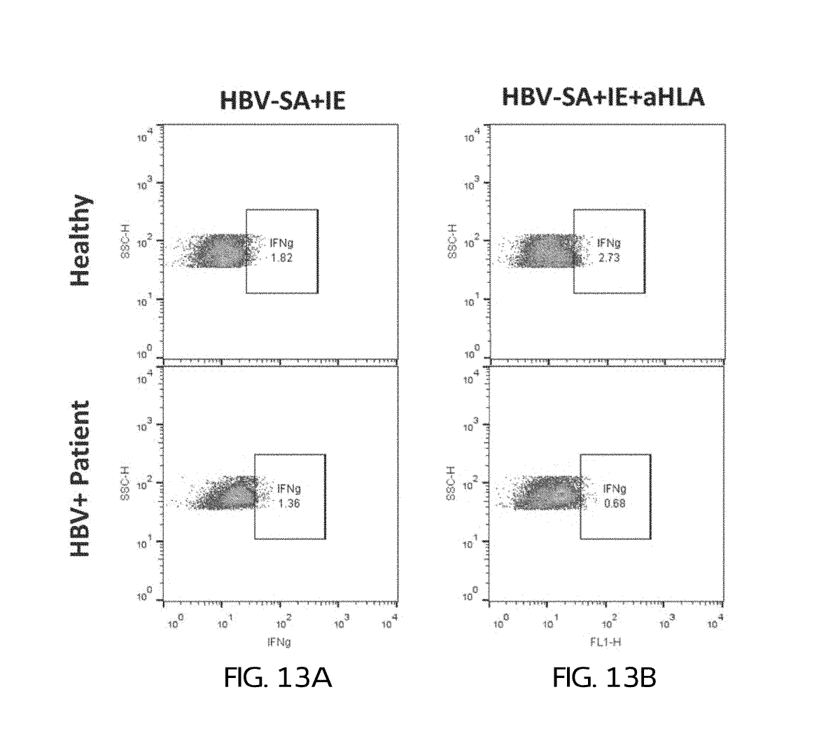

FIGS. 13A and 13B show the effect of HBV-SA plus IE (FIG. 13A) and HBV-SA plus IE plus anti-HLA class I antibody (aHLA) on the expression of interferon-gamma (IFNg) in PBMCs from healthy human subjects (top panels) and HBV patients (bottom panels).

In still another experiment, it was shown that low-dose IE induced interferon-gamma release from PBMCs, while medium-dose IE shifted the response toward IL-10 (an immunosuppressive cytokine) production and high-dose IE further shifted the response toward inducing apoptosis.

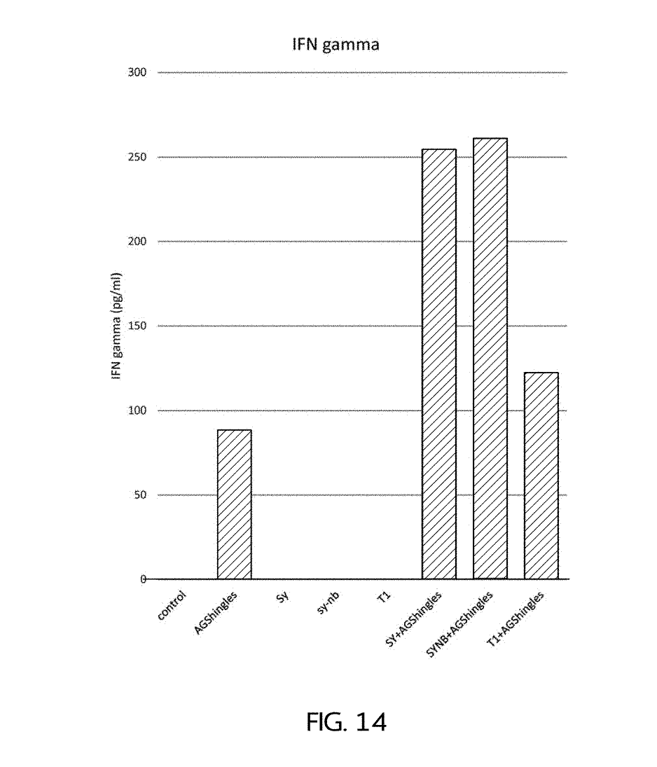

FIG. 14 shows the immune stimulation-enhancing activity of human alpha-synuclein protein (as measured by IFN-gamma release) for antigen challenge of whole blood from a subject recently immunized with the subject antigen. Whole blood for testing was obtained from a human subject recently immunized (approximately one week) with an approved shingles vaccine (i.e., immunized against Varicella zoster (chickenpox) virus antigens). Fresh whole blood aliquots were mixed with control and test protein/antigen compositions, and incubated at 37° C. for 24 hours. IFN-gamma release was then evaluated by ELISA assay. As shown in FIG. 14, the following controls and tests were performed: control (whole blood only; no added antigen or proteins); AGShingles (whole blood plus two antigens (Varicella zoster virus (VZV) ORF 26 recombinant protein at 3 μg/ml and Varicella zoster virus ORF 9 recombinant protein at 2 μg/ml, collectively at these concentrations “the AGShingles antigens”) present in the shingles vaccine the subject received); sy (whole blood plus boiled alpha-synuclein protein at 20 μg/ml); synb (whole blood plus alpha-synuclein protein not boiled at 20 μg/ml); T1 (whole blood plus heat-treated whole blood at 100 μg/ml); SY+AGShingles (whole blood plus boiled alpha-synuclein at 20 μg/ml and the AGShingles antigens; SYNB+AGShingles (whole blood plus not boiled alpha-synuclein at 20 μg/ml and the AGShingles antigens); and T1+AGShingles (whole blood plus T1 at 100 μg/ml and the AGShingles antigens). IFN-gamma release in the experiment is indicative of effector T-cell stimulation in the whole blood. As shown, control, sy, synb and T1 alone (i.e., all without added Varicella zoster virus antigen) did not cause IFN-gamma release. The Varicella zoster virus antigens alone caused IFN release (approximately 87 pg/ml). T1 plus the Varicella zoster virus antigens caused a moderately, further increased release of IFN-gamma (approximately 122 pg/ml). In contrast, both sy plus the Varicella zoster virus antigens and synb plus the Varicella zoster virus antigens caused a dramatic increase in IFN-gamma release (in each case to above 250 pg/ml). Thus, alpha-synuclein acts a potent enhancer of immune response against antigen.

The recombinant human HLA-B used in the experiments, which may also be used in the various embodiments, was cat# RPC140684-50 μg from Biomatik USA, LLC (Wilmington, Del., USA). The amino acid sequence of said HLA-B is shown in Table 1 (SEQ ID NO: 1).

TABLE 1
GSHSMRYFYTAIVISRPGRGEPRFISVGYVDDTQFVREDSDAASPREEPR
APWIEQEGPEYWDRNTQICKTNTQTYRESLRNLRGYYNQSEAGSHTLQRI
VIYGCDVGPDGRLLRGHDQYAYDGKDYIALNEDLSSWTAADTAAQITQRK
WEAAREAEQLRAYLEGLCVEWLRRYLENGKETLQRADPPKTHVTHHPISD
HEATLRCWALGFYPAEITLTWQRDGEDQTQDTELVETRPAGDRTFQKWAA
VVVPSGEEQRYTCHVQHEGLPKPLTLRWEPSSQSTIPI
(SEQ ID NO: 1)

The recombinant human HLA-G used in the experiments, which may also be used in the various embodiments, was cat# RPC140674-50 μg from Biomatik USA, LLC (Wilmington, Del., USA). The amino acid sequence of said HLA-G is shown in Table 2 (SEQ ID NO: 2).

TABLE 2
GSHSMRYFSAAVSRPGRGEPRFIAMGYVDDTQFVRFDSDSACPRMEPRAP
WVEQEGPEYWEEETRNTKAHAQTDRMNLQTLRGYYNQSEASSHTLQWMIG
CDLGSDGRLLRGYEQYAYDGKDYLALNEDLRSWTAADTAAQISKRKCEAA
NVAEQRRAYLEGTCVEWLHRYLENGKEMLQRADPPKTHVTHHPVFDYEAT
LRCWALGFYPAEIILTWQRDGEDQTQDVELVETRPAGDGTFQKWAAVVVP
SGEEQRYTCHVQHEGLPEPLMLRWKQSSLPTIPIMGIVAGLVVLAAVVTG
AAVAAVLWRKKSS
(SEQ ID NO: 2)

The recombinant human alpha-synuclein used in the experiments, which may also be used in the various embodiments, was cat # PRO-393 from ProSpec-Tany TechnoGene Ltd. (“ProspecBio;” East Brunswick, N.J., USA). The amino acid sequence of said human alpha-synuclein is shown in Table 3 (SEQ ID NO: 3). Varicella Zoster Virus ORF 26 recombinant protein used in the experiments was ProspecBio cat# Pro-233 and Varicella Zoster Virus ORF 9 recombinant protein used in the experiments was ProspecBio cat# Pro-232.

TABLE 3
MDVFMKGLSKAKEGVVAAAEKTKQGVAEAAGKTKEGVLYVGSKTKEGVVH
GVATVAEKTKEQVTNVGGAVVTGVTAVAQKTVEGAGSIAAATGFVKKDQL
GKNEEGAPQEGILEDMPVDPDNEAYEMPSEEGYQDYEPEA
(SEQ ID NO: 3)

The ability of red blood cells to augment immune responses has been previously described, especially with respect to immunosuppression arising from red blood cell transfusions. Speculation for the elements that might be responsible on the surface of red blood cells for this effect have led to studies of LFA-3, a protein that is highly enriched in red blood cells. With regard to immune processes, LFA-3 is a ligand on antigen-presenting cells (APCs) that interacts with the CD2 receptor on CD4+ cells and is thought to be a co-activator that works in conjunction with the interaction between HLAs and TCRs on T-cells. It should be pointed out that the LFA-3 in a blood preparation is in the context of being present on APCs and not as a free ligand. Nevertheless, soluble LFA-3 was tested (in the same manner as alpha-synuclein) for an ability to induce an antigen-specification stimulation/modulation. The results showed that the presence of LFA-3 had no effects on immune responses in PBMCs exposed to antigen. Thus, it was determined that, in contrast to alpha-synuclein, LFA-3 in solution does not have antigen-specific immune-modulating activity.

Without limitation, the invention also provides the following enumerated embodiments.

### Embodiment 1

An immunomodulatory composition, such as an immunomodulatory pharmaceutical composition, including a mixture of:

(i) a first component including
(a) at least partially purified HLA protein or fragments thereof, such as mammalian, for example human,
(b) whole blood, such as mammalian, for example human, or a cellular fraction thereof, such as a density gradient fraction/layer thereof, such as but not limited to a white blood cell and/or red blood cell (erythrocyte) and/or platelet fraction/layer thereof, or a cell membrane fraction/preparation of any of the foregoing or a protein extract of any of the foregoing, and/or
(c) alpha-synuclein protein, such as mammalian alpha-synuclein protein, such as human alpha-synuclein protein, or fragments thereof; and

(ii) at least one preselected antigen or immunogen, such as at least one preselected peptide antigen, at least one preselected protein antigen, at least one preselected carbohydrate antigen, at least one preselected lipid antigen, and/or at least one preselected glycolipid antigen. For example, the at least one preselected antigen or immunogen may be other than an HLA protein or fragment thereof and/or other than a synuclein protein or fragment thereof, such as other than a mammalian alpha-synuclein protein or fragment thereof.

### Embodiment 2

The immunomodulatory composition of embodiment 1, in which the first component, such as at least partially purified HLA protein, is heat-treated, for example, heat-treated at or above 100° C. for at least 1 or 2 or 3 or 4 or 5 minutes.

### Embodiment 3

The immunomodulatory composition of embodiment 1, in which the at least partially purified HLA protein is at least substantially denatured.

### Embodiment 4

The immunomodulatory composition of any one of the preceding embodiments, in which the first component includes at least partially purified HLA protein is recombinant, such as full length or partial length recombinant protein.

### Embodiment 5

The immunomodulatory composition of embodiments 1-3, in which the at least partially purified HLA protein is derived from a tissue source.

### Embodiment 6

The immunomodulatory composition of embodiment 5, in which the tissue source includes blood cells.

### Embodiment 7

The immunomodulatory composition of embodiment 5, in which the tissue source at least substantially or at least predominantly includes red blood cells.

### Embodiment 8

The immunomodulatory composition of any one of the preceding embodiments, in which the at least one preselected peptide antigen includes a synthetic peptide. The peptide may, for example, be 5-20 amino acids in length or any subrange thereof or number of amino acids therein.

### Embodiment 9

The immunomodulatory composition of any one of the preceding embodiments, in which the at least one preselected antigen includes a self-antigen.

### Embodiment 10

The immunomodulatory composition of embodiment 9, in which the self-antigen is associated with an autoimmune disease.

### Embodiment 11

The immunomodulatory composition of any one of embodiments 1-8, in which the at least one preselected antigen is a cancer-associated antigen or an antigen preferentially expressed on cancer cells versus normal cells.

### Embodiment 12

The immunomodulatory composition of any one of the preceding embodiments, in which the at least partially purified HLA protein includes at least partially purified mammalian HLA protein.

### Embodiment 13

The immunomodulatory composition of embodiment 12, in which the at least partially purified mammalian HLA protein includes at least partially purified human HLA protein.

### Embodiment 14

The immunomodulatory composition of any one of the preceding embodiments, in which the composition is in a form selected from the group consisting of a liquid form and an at least substantially dry form, such as a powder form or tableted form. A dry form may, for example, be obtained by lyophilizing or otherwise drying a liquid mixture of the components.

### Embodiment 15

The immunomodulatory composition of embodiment 14, in which the composition is a parenteral composition.

### Embodiment 16

The immunomodulatory composition of embodiment 15, in which the composition is an injectable composition.

### Embodiment 17

The immunomodulatory composition of any one of the preceding embodiments, in which the at least partially purified HLA protein includes one or more of HLA-A, HLA-B, HLA-C and HLA-G protein.

### Embodiment 18

The immunomodulatory composition of embodiment 17, in which the at least partially purified HLA protein includes HLA-G protein.

### Embodiment 19

The immunomodulatory composition of any one of the preceding embodiments, in which the at least partially purified HLA protein includes HLA Class II protein.

### Embodiment 20

A method for manufacturing an immunomodulatory composition, such as an immunomodulatory pharmaceutical composition, including the steps of:

providing a first component including
(a) at least partially purified HLA protein or fragments thereof, such as mammalian, for example human,
(b) whole blood, such as mammalian, for example human, or a cellular fraction thereof, such as a density gradient fraction thereof, such as but not limited to a white blood cell and/or red blood cell (erythrocytes) and/or platelet fraction/layer thereof, or a cell membrane fraction/preparation of any of the foregoing or a protein extract of any of the foregoing, and/or
(c) alpha-synuclein protein, such as mammalian alpha-synuclein protein, such as human alpha-synuclein protein, or fragments thereof; and

providing a second component including at least one preselected antigen or immunogen, such as at least one preselected peptide antigen, at least one preselected protein antigen, at least one preselected carbohydrate antigen, at least one preselected lipid antigen, and/or at least one preselected glycolipid antigen; and

mixing the first component and the second component, for example, under aqueous conditions.

### Embodiment 21

The method of embodiment 20, in which the at least partially purified HLA protein is provided and the method further includes the step of:

heat-treating the at least partially purified HLA protein before the mixing step.

### Embodiment 22

The method of embodiment 20, in which at least partially purified HLA protein is provided and the method further includes the step of:

denaturing the at least partially purified HLA protein before the mixing step.

### Embodiment 23

The method of embodiment 20, further including the step of:

heat-treating the composition after the mixing step.

### Embodiment 24

The method of embodiment 20, in which

the providing step includes providing said fragments, and

the mixing step includes mixing said fragments with said second component.

### Embodiment 25

The method of any one of embodiments 20-24, in which the at least partially purified HLA protein includes recombinant HLA protein.

### Embodiment 26

The method of any one of embodiments 20-24, in which the at least partially purified HLA protein is derived from a tissue source.

### Embodiment 27

The method of embodiment 26, in which the tissue source includes blood cells.

### Embodiment 28

The method of embodiment 27, in which the tissue source at least substantially includes red blood cells.

### Embodiment 29

The method of any one of embodiments 20-28, in which the at least one preselected antigen includes a synthetic peptide.

### Embodiment 30

The method of any one of embodiments 20-29, in which the at least one preselected antigen includes a self-antigen.

### Embodiment 31

The method of embodiment 30, in which the self-antigen is associated with an autoimmune disease.

### Embodiment 32

The method of any one of embodiments 20-29, in which the at least one preselected antigen, which may, for example, be or include one or more synthetic peptides, includes a cancer-associated antigen/epitope or an antigen/epitope preferentially expressed on cancer cells versus normal cells.

### Embodiment 33

The method of any one of embodiments 20-29, in which the at least one preselected peptide antigen includes an antigen of a virus or cellular microorganism, such as a pathogenic virus or cellular microorganism, for example for a mammal such as human. Without limitation, the at least one preselected antigen of a pathogenic virus may, for example, be or include an antigen of or associated with Hepatitis B virus, Hepatitis C virus, Influenza virus, HIV-1 or HIV-2. For example, the virus may be Hepatitis B and the at least one preselected antigen may be one or more of HBsAg (surface antigen, S-protein) such as SEQ ID NO: 37 (adw serotype) and/or SEQ ID NO: 38 (adr serotype), HB pre-S1 protein (SEQ ID NO: 39), HB pre-S2 protein (SEQ ID NO: 40), HBeAg (HepB envelope antigen; e.g., SEQ ID NO: 41), and HBcAg (HepB core antigen; e.g. SEQ ID NO: 42). Without limitation the at least one preselected antigen of a pathogenic cellular microorganism may, for example, be or include an antigen of or associated with a pathogenic bacteria, fungi, protozoan, or amoeba.

### Embodiment 34

The method of any one of embodiments 20-33, in which the at least partially purified HLA protein includes one or more of HLA-A, HLA-B, HLA-C and HLA-G protein.

### Embodiment 35

The method of embodiment 34, in which the at least partially purified HLA protein includes HLA-G.

### Embodiment 36

The method of any one of embodiments 20-35, in which the at least partially purified HLA protein includes HLA Class II protein.

### Embodiment 37

A method for modulating the immune response in a mammal to at least one preselected antigen or immunogen including administering to the mammal the immunomodulatory pharmaceutical composition of any one of embodiments 1-19. Said administration may be parenteral or non-parenteral. Said administration may, for example be via ingestion. Where administration is via ingestion, an antacid may be co-administered. The composition may, for example, be an enteric composition for ingestion. Administration may, for example, be via injection, such as intravenous injection, intra-thymic injection or injection into a lymph node of a subject.

### Embodiment 38

The method of embodiment 36, in which said administration is parenteral.

### Embodiment 39

The method of embodiment 36, in which said administration is via injection.

### Embodiment 40

The method of any one of embodiments 36-38, in which the mammal is a human.

### Embodiment 41

The method of any one of embodiments 36-39, in which the resultant modulation of the immune response is immunosuppressive (pro-regulatory cell response) with respect to the at least one preselected antigen or immunogen. Thus, alpha-synuclein may be used as a pro-regulatory (-immunosuppressive) response adjuvant.

### Embodiment 42

The method of any one of embodiments 36-39, in which the resultant modulation of the immune response is immunostimulatory (pro-effector cell response) with respect to the at least one preselected antigen or immunogen. Thus, alpha-synuclein may be used as a pro-effector response adjuvant.

### Embodiment 43

Use of a composition according to any one of embodiments 1-19 for modulating the immune response in a mammal, such as a human, to the at least one preselected antigen or immunogen.

### Embodiment 44

The use of embodiment 43, in which the modulation of the immune response is immunosuppressive with respect to the at least one preselected antigen or immunogen.

### Embodiment 45

The use of embodiment 43, in which the modulation of the immune response is immunostimulatory with respect to the at least one preselected antigen or immunogen.

### Embodiment 46

Use of alpha-synuclein protein, such as mammalian alpha-synuclein protein, such as human alpha-synuclein protein, or fragments thereof, as an immune stimulator or adjuvant in conjunction with a vaccination (use of a vaccine), for example, in a mammal such as but not limited to a human, such as, in conjunction with vaccination against a pathogen or a cancer antigen or in conjunction with use of a cancer vaccine.

### Embodiment 47

Use of alpha-synuclein protein, such as mammalian alpha-synuclein protein, such as human alpha-synuclein protein, or fragments thereof, as an immunization adjuvant or vaccine adjuvant, for example, in a mammal such as but not limited to a human.

### Embodiment 48

A method for enhancing the immune response to an immunization with an immunogen in a subject such as a mammal, such as but not limited to a human, comprising the step of: coadministering to the subject alpha-synuclein protein, such as mammalian alpha-synuclein protein, such as human alpha-synuclein protein, or fragments thereof, with the immunogen.

### Embodiment 49

A method for enhancing the immune response of a mammalian subject, such as but not limited to a human, having a malignancy, such as a blood cancer/malignancy or a solid tumor, to said malignancy and/or a method for treating such a malignancy in a such a subject, comprising the step of: administering to the subject alpha-synuclein protein, such as mammalian alpha-synuclein protein, such as human alpha-synuclein protein, or fragments thereof. Said administration may be with or without at least one preselected antigen. Said blood cancer/malignancy may, for example, be Myelodysplastic syndrome (MDS), a leukemia, such as Acute lymphoblastic leukemia (ALL) or Acute myeloid leukemia (AML), or a lymphoma, such as a Hodgkin lymphoma, non-Hodgkin lymphoma or mantle cell lymphoma. Said malignancy may, for example, be a liver cancer such as hepatocellular carcinoma (HCC) or cholangiocarcinoma, pancreatic cancer, breast cancer, prostate cancer, kidney cancer, melanoma, myeloma, glioblastoma, ovarian cancer, testicular cancer, bone cancer such as osteosarcoma, or lung cancer such as non-small cell lung cancer or small cell lung cancer.

### Embodiment 50

Use of alpha-synuclein protein, such as mammalian alpha-synuclein protein, such as human alpha-synuclein protein, or fragments thereof, in the treatment of a malignancy/cancer, such as a blood cancer or a solid tumor, in a mammal such as but not limited to a human. Said administration may be with or without at least one preselected antigen. Said blood cancer/malignancy may, for example, be Myelodysplastic syndrome (MDS), a leukemia, such as ALL or AML, or a lymphoma, such as a Hodgkin lymphoma, non-Hodgkin lymphoma or mantle cell lymphoma. Said malignancy may, for example, be a liver cancer such as hepatocellular carcinoma (HCC) or cholangiocarcinoma, pancreatic cancer, breast cancer, prostate cancer, kidney cancer, melanoma, myeloma, glioblastoma, ovarian cancer, testicular cancer, bone cancer such as osteosarcoma, or lung cancer such as non-small cell lung cancer or small cell lung cancer.

### Embodiment 51

Use of alpha-synuclein protein, such as mammalian alpha-synuclein protein, such as human alpha-synuclein protein, or fragments thereof, in the preparation of a medicament for the treatment of a malignancy, such as a blood cancer or a solid tumor, in a mammal such as but not limited to a human. Said medicament may include or exclude at least one preselected antigen as described herein. Said blood cancer/malignancy may, for example, be Myelodysplastic syndrome (MDS), a leukemia, such as ALL or AML, or a lymphoma, such as a Hodgkin lymphoma, non-Hodgkin lymphoma or mantle cell lymphoma. Said malignancy may, for example, be a liver cancer such as hepatocellular carcinoma (HCC) or cholangiocarcinoma, pancreatic cancer, breast cancer, prostate cancer, kidney cancer, melanoma, myeloma, glioblastoma, ovarian cancer, testicular cancer, bone cancer such as osteosarcoma, or lung cancer such as non-small cell lung cancer or small cell lung cancer.

### Embodiment 52

A pharmaceutical composition for the treatment of a malignancy, such as a blood cancer or a solid tumor, in a mammal such as but not limited to a human, said composition comprising a therapeutically effective amount of alpha-synuclein protein, such as mammalian alpha-synuclein protein, such as human alpha-synuclein protein, or fragments thereof. The composition may further include one or more pharmaceutically acceptable excipients. Said blood cancer/malignancy may, for example, be Myelodysplastic syndrome (MDS), a leukemia, such as ALL or AML, or a lymphoma, such as a Hodgkin lymphoma, non-Hodgkin lymphoma or mantle cell lymphoma. Said malignancy may, for example, be a liver cancer such as hepatocellular carcinoma (HCC) or cholangiocarcinoma, pancreatic cancer, breast cancer, prostate cancer, kidney cancer, melanoma, myeloma, glioblastoma, ovarian cancer, testicular cancer, bone cancer such as osteosarcoma, or lung cancer such as non-small cell lung cancer or small cell lung cancer.

### Embodiment 53

A method for enhancing the immune response of a mammalian subject, such as but not limited to a human, having an infectious disease, such as a microbial or viral infection, to said infectious disease, comprising the step of: administering to the subject alpha-synuclein protein, such as mammalian alpha-synuclein protein, such as human alpha-synuclein protein, or fragments thereof. Said administration may be with or without at least one preselected antigen as described herein.

### Embodiment 54

Use of alpha-synuclein protein, such as mammalian alpha-synuclein protein, such as human alpha-synuclein protein, or fragments thereof, in the treatment of an infectious disease, such as a microbial or viral infection, in a mammal such as but not limited to a human. Said use may be in combination with or exclude at least one preselected antigen as described herein.

### Embodiment 55

Use of alpha-synuclein protein, such as mammalian alpha-synuclein protein, such as human alpha-synuclein protein, or fragments thereof, in the preparation of a medicament, such as a medicament for the treatment of an infectious disease, such as a microbial or viral infection, in a mammal such as but not limited to a human. Said medicament may include or exclude at least one preselected antigen as described herein.

### Embodiment 56

A pharmaceutical composition for the treatment of an infectious disease, such as a microbial or viral infection, in a mammal such as but not limited to a human, said composition comprising a therapeutically effective amount of alpha-synuclein protein, such as mammalian alpha-synuclein protein, such as human alpha-synuclein protein, or fragments thereof. The composition may further include one or more pharmaceutically acceptable excipients. The composition may include or exclude at least one preselected antigen as described herein. In one example, the viral infection is Hepatitis B. In a related example, the viral infection is Hepatitis B and the composition includes at least one Hepatitis B protein or peptide antigen such as but not limited to one or more of HBsAg (surface antigen, S-protein) such as SEQ ID NO: 37 (adw serotype) and/or SEQ ID NO: 38 (adr serotype), HB pre-S1 protein (SEQ ID NO: 39), HB pre-S2 protein (SEQ ID NO: 40), HBeAg (HepB envelope antigen; e.g., SEQ ID NO: 41), and HBcAg (HepB core antigen; e.g. SEQ ID NO: 42).

### Embodiment 57

An immunomodulatory composition, such as an immunomodulatory pharmaceutical composition, including, for example as or in a mixture:

(a) at least substantially pure alpha-synuclein protein or a fragment thereof; and

(b) at least one preselected antigen, such as a peptide antigen, or immunogen, which antigen or immunogen is not a human or non-human mammalian alpha-synuclein protein or a fragment thereof.

In one variation, the at least one preselected antigen or at least one preselected immunogen does not comprise a synuclein protein and/or does not comprise a fragment of a synuclein protein.

### Embodiment 58

The immunomodulatory composition of embodiment 57, further including at least partially purified human or non-human mammalian HLA protein or fragments thereof, wherein the at least one preselected antigen or immunogen does not include human or non-human mammalian HLA protein or fragments thereof.

### Embodiment 59

The immunomodulatory composition of embodiment 57, wherein the composition does not include HLA protein or fragments thereof.

### Embodiment 60

Any of composition embodiments 57-59, further including at least one pharmaceutically acceptable excipient.

### Embodiment 61

A method for modulating the immune response in a mammal to at least one preselected antigen or immunogen including administering to a human or non-human mammal a immunomodulatory pharmaceutical composition according to any one of embodiments 57-60.

### Embodiment 62

A method for modulating the immune response in a mammal to at least one preselected antigen or immunogen including co-administering to a human or non-human mammal

(a) at least substantially pure alpha-synuclein protein or a fragment thereof; and

(b) at least one preselected antigen or immunogen, which is not a human or non-human mammalian alpha-synuclein protein or a fragment thereof. In one variation, the at least one preselected antigen or at least one preselected immunogen does not comprise a synuclein protein and/or does not comprise a fragment of a synuclein protein.

### Embodiment 63

The method embodiment 62, further including co-administering at least partially purified human or non-human mammalian HLA protein or fragments thereof to the human or non-human mammal, wherein the at least one preselected antigen or immunogen does not include human or non-human mammalian HLA protein or fragments thereof.

### Embodiment 64

The method embodiment 62, wherein HLA protein or fragments thereof are not co-administered to the human or non-human mammal.

### Embodiment 65

A method for manufacturing an immunomodulatory composition, such as an immunomodulatory pharmaceutical composition, including the steps of:

providing at least substantially purified human or non-human mammalian alpha-synuclein protein or a fragment thereof;

providing at least one preselected antigen or immunogen, which is not a human or non-human mammalian alpha-synuclein protein or a fragments thereof; and

mixing the at least substantially purified mammalian alpha-synuclein protein or fragments thereof and the at least one preselected antigen or immunogen.

### Embodiment 66

The method embodiment 65, further including:

providing at least partially purified human or non-human mammalian HLA protein or fragments thereof, and

wherein said mixing step further includes mixing the provided at least substantially pure alpha-synuclein protein or fragments thereof, the at least one preselected antigen or immunogen, and the at least partially purified human or non-human mammalian HLA protein or fragments thereof, and

wherein the at least one preselected antigen or immunogen is not a human or non-human mammalian HLA protein or fragment thereof.

In one variation, the at least one preselected antigen or immunogen is not an HLA protein or fragment thereof.

### Embodiment 67

Either of embodiments 65 and 66, further including providing at least one pharmaceutically acceptable excipient, and mixing said at least one pharmaceutical excipient with the other mix components of said embodiments.

### Embodiment 68

A pharmaceutical composition including a mammalian alpha-synuclein protein, such as human alpha-synuclein protein, or at least one fragment thereof, such as a substantial fragment thereof. The alpha-synuclein protein or fragment thereof may be recombinant or synthetic. In one variation, the pharmaceutical composition consists essentially of a mammalian alpha-synuclein, such as human alpha-synuclein protein, or a substantial fragment thereof. In another variation, the pharmaceutical composition consists essentially of a mammalian alpha-synuclein, such as human alpha-synuclein protein, or a substantial fragment thereof and at least one preselected antigen or immunogen. The compositions may further include one or more pharmaceutically-acceptable excipients. The compositions may be parenteral or non-parenteral formulations. The compositions may, for example, be oral pharmaceutical compositions (formulated for oral administration (ingestion)) or in formulated in any manners described in this disclosure. The compositions may be in a solid dosage form, such as a powder, tablet or capsule. The compositions may be in a liquid form for either parenteral or non-parental administration. The compositions may be in liquid form for administration by injection.

### Embodiment 69

Use of a mammalian alpha-synuclein protein, such as human alpha-synuclein protein, or at least one fragment thereof, such as a substantial fragment thereof, as a medicament, for example in mammals such as humans. The alpha-synuclein protein or fragment thereof may be recombinant or synthetic, or purified from a tissue source.

The at least one preselected antigen or immunogen of various embodiments may, for example, be or include a peptide such as a synthetic peptide. The at least one preselected antigen or immunogen may, for example, be or include a protein such as an at least substantially purified protein, such as but not limited to an at least substantially purified recombinant protein. The at least one preselected antigen or immunogen may be or include a recombinant protein or a recombinant protein fragment of a full-length protein. Wherever throughout this disclosure an embodiment refers to a peptide antigen, it should be understood that corresponding embodiments directed to protein antigens are also intended to be disclosed and vice versa.

The at least one preselected peptide/protein antigen or immunogen may, for example, be or include a self-antigen of a human or non-human mammal, for example, a self-antigen associated with an autoimmune disease. The at least one preselected peptide/protein antigen or immunogen may, for example, be or include a cancer-associated antigen or an antigen preferentially expressed on cancer cells versus normal cells of a human or non-human mammal. The at least one preselected peptide/protein antigen may, for example, be or include a viral antigen, for example, of a pathogenic virus, such as a pathogenic virus that currently infects the subject or has previously infected the subject. For example, the virus may be Hepatitis B and the at least one preselected antigen may be one or more of HBsAg (surface antigen, S-protein) such as SEQ ID NO: 37 (adw serotype) and/or SEQ ID NO: 38 (adr serotype), HB pre-S1 protein (SEQ ID NO: 39), HB pre-S2 protein (SEQ ID NO: 40), HBeAg (HepB envelope antigen; e.g., SEQ ID NO: 41), and HBcAg (HepB core antigen; e.g. SEQ ID NO: 42).

The at least one preselected antigen that is or includes a self-antigen may be or include S-antigen, such as human S-antigen (SEQ ID NO: 4 or SEQ ID NO: 5) and/or a protein or peptide fragment thereof such as a synthetic peptide fragment. Immunomodulatory pharmaceutical composition embodiments of the invention including such antigens may be administered for the treatment of uveitis or AMD in a human or non-human mammal. In a particular embodiment, the peptide is or includes a fragment of S-antigen such as GEPIPVTVDVTNNTEKTVKK (SEQ ID NO: 6) or VTVDVTNNTEKTVKK (SEQ ID NO: 7). Other synthetic peptides derived from or related to human S-antigen that may be used include:

| | | |
|---|---|---|
| B27PD: | ALNED LSSWT AADT | (SEQ ID NO: 8) |
| Peptide 2 | IFKKI SRDKS VTIYL | (SEQ ID NO: 9) |
| (P2): | | |
| Peptide 6 | VKGKK VYVTL TCAFR | (SEQ ID NO: 10) |
| (P6): | | |
| Peptide 8 | YGQED VDVIG LTFRR | (SEQ ID NO: 11) |
| (P8): | | |
| Peptide 29 | LPLLA NNRER RGIAL | (SEQ ID NO: 12) |
| (P29): | | |
| Peptide 31 | DTNLA SSTII KEGID | (SEQ ID NO: 13) |
| (P31): | | |
| PDS-Ag: | FLGEL TSSEV ATEV | (SEQ ID NO: 14) |

The peptides may, for example, be the only preselected antigens or they may be used in any combination in the immunomodulatory compositions.

In certain embodiments, the patient has early AMD, characterized by medium drusen (63-125 μm) without pigmentary abnormalities thought to be related to AMD. In other embodiments, the patient has intermediate AMD, characterized by large drusen or with pigmentary abnormalities associated with at least medium drusen. In still other embodiments, the patient has late AMD, characterized by lesions associated with neovascular AMD or geographic atrophy. Drusen, which are yellow or white accumulations of extracellular material that build up between Bruch's membrane and the retinal pigment epithelium of the eye, can be measured by any technique known by the skilled artisan. In certain embodiments, drusen volumes are measured by spectral domain optical coherence tomography (SD-OCT). In other embodiments, the patient has wet AMD which may be associated with choroidal neovascularization (CNV).

A related embodiment provides a method for treating AMD in a human or non-human mammalian subject that includes administering any of said immunomodulatory pharmaceutical compositions to the subject. In various embodiments, the result obtained by treatment of AMD or uveitis includes cessation and/or slowing of disease progression, for example, progression from early AMD to intermediate AMD, or progression from intermediate AMD to late AMD, or cessation or slowing of progression to wet AMD, or cessation and/or slowing of neovascularization in wet AMD.

Another embodiment of the invention provides immunomodulatory pharmaceutical compositions according to the invention for the treatment of multiple sclerosis in a mammalian subject, such as a human patient, in which the at least one preselected antigen is or includes myelin basis protein (MBP) such as human myelin basis protein (for example, Genbank Accession No. AAC41944 myelin basic protein [Homo sapiens] SEQ ID NO: 15 (see also amino acid sequence Table 4)) and/or one or more fragments thereof, such as synthetic peptides. A related embodiment provides a method for treating multiple sclerosis in a human or non-human mammalian subject that includes administering said immunomodulatory composition to the subject.

TABLE 4

| | |
|---|---|
| 1 | masqkrpsqr hgskylatas tmdharhgfl prhrdtgild sigrffggdr gapkrgsgkv |
| 61 | pwlkpgrspl psharsqpgl cnmykdshhp artahygslp qkshgrtqde npvvhffkni |
| 121 | vtprtpppsq gkgrglslsr fswgaegqrp gfgyggrasd yksahkgfkg vdaqgtlski |
| 181 | fklggrdsrs gspmarrhhh hhh (SEQ ID NO: 15) |

Another embodiment of the invention provides an immunomodulatory pharmaceutical composition according to the invention for the treatment of rheumatoid arthritis in a human or non-human mammalian subject in which the at least one preeletced antigen of the composition is or includes type II collagen such as human type II collagen protein (for example, Genbank Accession No. AAC41772 alpha-1 type II collagen [Homo sapiens]; SEQ ID NO:16 (see also amino acid sequence Table 5)) and/or one or more peptide fragments thereof, such as synthetic peptides. A related embodiment provides a method for treating rheumatoid arthritis in a human or non-human mammalian subject that includes administering said immunomodulatory composition to the subject.

TABLE 5

| | |
|---|---|
| 1 | mirlgapqsl vlltllvaav lrcqgqdvqe agscvqdgqr yndkdvwkpe pericvcdtg |
| 61 | tvlcddiice dvkdclspei pfgeccpicp tdlatasgqp gpkgqkgepg dikdivgpkg |
| 121 | ppgpqgpage qgprgdrgdk gekgapgprg rdgepgtpgn pgppgppgpp gppglggnfa |
| 181 | aqmaggfdek aggaqlgvmq gpmgpmgprg ppgpagapgp qgfqgnpgep gepgvsgpmg |
| 241 | prgppgppgk pgddgeagkp gkagergppg pqgargfpgt pglpgvkghr gypgldgakg |
| 301 | eagapgvkge sgspgengsp gpmgprglpg ergrtgpaga agargndgqp gpagppgpvg |
| 361 | paggpgfpga pgakgeagpt gargpegaqg prgepgtpgs pgpagasgnp gtdgipgakg |
| 421 | sagapgiaga pgfpgprgpp gpqgatgplg pkgqtgepgi agfkgeqgpk gepgpagpqg |
| 481 | apgpageegk rgargepggv gpigppgerg apgnrgfpgq dglagpkgap gergpsglag |
| 541 | pkgangdpgr pgepglpgar gltgrpgdag pqgkvgpsga pgedgrpgpp gpqgargqpg |
| 601 | vmgfpgpkga ngepgkagek glpgapglrg lpgkdgetga agppgpagpa gergeqgapg |
| 661 | psgfqglpgp pgppgeggkp gdqgvpgeag apglvgprge rgfpgergsp gaqglqgprg |
| 721 | lpgtpgtdgp kgasgpagpp gaqgppglqg mpgergaagi agpkgdrgdv gekgpegapg |
| 781 | kdggrgltgp igppgpagan gekgevgppg pagsagarga pgergetgpp gpagfagppg |
| 841 | adgqpgakge qgeagqkgda gapgpqgpsg apgpqgptgv tgpkgargaq gppgatgfpg |
| 901 | aagrvgppgs ngnpgppgpp gpsgkdgpkg argdsgppgr agepglqgpa gppgekgepg |
| 961 | ddgpsgaegp pgpqglagqr givglpgqrg ergfpglpgp sgepgkqgap gasgdrgppg |
| 1021 | pvgppgltgp agepgregsp gadgppgrdg aagvkgdrge tgavgapgap gppgspgpag |
| 1081 | ptgkqgdrge agaqgpmgps gpagargiqg pqgprgdkge agepgerglk ghrgftglqg |
| 1141 | lpgppgpsgd qgasgpagps gprgppgpvg psgkdgangi pgpigppgpr grsgetgpag |
| 1201 | ppgnpgppgp pgppgpgidm safaglgpre kgpdplqymr adqaagglrq hdaevdatlk |
| 1261 | slnnqiesir spegsrknpa rtcrdlklch pewksgdywi dpnqgctlda mkvfcnmetg |
| 1321 | etcvypnpan vpkknwwssk skekkhiwfg etinggfhfs ygddnlapnt anvqmifirl |
| 1381 | lstegsqnit yhcknsiayl deaagnlkka lliqgsndve iraegnsrft ytalkdgctk |
| 1441 | htgkwgktvi eyrsqktsrl piidiapmdi ggpeqefgvd igpvcfl (SEQ ID NO: 16) |

Still further provided are immunomodulatory composition and a method embodiments for the amelioration of treatment-limiting immune reactivity in a mammalian subject, such as a human patient, that develops against a therapeutic protein that has been administered to the subject, such as a therapeutic antibody, e.g., a monoclonal antibody, such as Herceptin® (trastuzumab) or Avastin® (bevacizumab), or a soluble receptor, a growth factor, or an enzyme such as in enzyme replacement therapy. In this case, the at least one preselected antigen of the composition and method embodiments may, for example, be or include the therapeutic protein or one or more fragments thereof, or one or more peptides representing at least a portion of the amino acid sequence of the therapeutic protein.

At least partially purified HLA protein may, for example, be or include at least partially purified mammalian HLA protein. At least partially purified mammalian HLA protein may, for example, be or include at least partially purified human HLA protein. At least partially purified HLA protein may, for example, be or include one or more of HLA-A, HLA-B, HLA-C and HLA-G protein. At least partially purified HLA protein may, for example, be or include HLA Class II protein.

Alpha-synuclein and/or HLA and/or any proteins of embodiments of the invention may, for example, be recombinant or may be purified from tissue.

The immunomodulatory pharmaceutical composition may, for example, be in liquid form or in a solid/dry form such as in a powder or tablet form. The immunomodulatory pharmaceutical compositions may be parenteral or non-parenteral compositions. The immunomodulatory pharmaceutical compositions may, for example, be injectable compositions such as a liquid, for example aqueous, injectable solution or suspension. The immunomodulatory pharmaceutical compositions may, for example, be orally administrable compositions. Administration to a subject may be by any route, such as parenteral or non-parenteral or a combination of routes. Administration may, for example, be made via injection or oral administration (ingestion) or by direct delivery to any part/section of the alimentary canal. Solid pharmaceutical compositions for oral administration via ingestion such as tablets or capsule may, for example, be enteric coated or otherwise formulated to prevent or minimize dissolution in the stomach but allow dissolution in the small intestine. Compositions for oral administration via ingestion may, for example, comprise or be co-administered with an antacid or other acid-reducing agent, such as omeprazole.

The resultant modulation of the immune response may be immunosuppressive, e.g., at least partially tolerance-inducing, with respect to the at least one preselected antigen or immunogen, or the resultant modulation of the immune response is immunostimulatory with respect to the at least one preselected antigen or immunogen.

In a variation of any of the embodiments presented herein, the composition or mixture excludes (does not include) beta-2 microglobulin.

Still further embodiments of the invention are directed to methods and compositions for preventing and/or treating Hepatitis B infections, such as chronic Hepatitis B infections, Hepatitis B-associated liver diseases and/or Hepatitis B-associated cancers such as hepatocellular carcinoma (HCC), in non-human mammals and human patients.

Two studies were previously carried out with oral administration of HB SAg for treatment of patients with chronic Hepatitis B virus (“HBV,” “HepB”) infection. In Safadi et al. 2003 (Am J Gastroenterology 98: 2505-2515), a mixture of HB SAg+preS1+preS2 proteins was administered 3 times a week to a total of 42 chronic HBV patients. A significant drop in viral loads was seen for 15 out of 49 patients, and HB SAg and HBcAg biopsy scores were improved in 41% and 57% respectively. More interestingly, among the patients treated, 19 were HBeAg positive, the significance being that the presence of this marker is an indication that the patient has a higher risk for development of hepatocellular carcinoma (reviewed in Sharma et al. 2005). One criteria for successful treatment is loss of this marker and indeed, out of the 19 HBeAg patients treated, 5 of them turned HBeAg negative and 4 of these 5 developed anti-HBeAg antibodies, thereby converting from what is termed a chronic carrier into an inactive carrier. Inactive carriers are considered to be in an essentially benign infected state associated with only a very low propensity for developing hepatocellular carcinoma (Sharma et al. 2005). In addition, another characteristic of the potential for cancer development over time is the change in a patient's profile where Th1 responses are reduced and Th2 responses increase. The effects of the oral treatment described in Safadi et al. resulted in 17/27 patients showing an increases in IFN-gamma secretion (an increase in a Th1 response) and 13/27 patients showing a reduction of IL10 secretion (a decrease in a Th2 response) thus showing a reversal in markers for progression towards development of hepatocellular carcinoma. In addition, 21/27 of the patients showed an increased HB SAg specific T cell proliferation, a potentially further indication that the recipients were mounting an effective Th1 response to HBV.

In a similar but separate study that was part of a limited clinical trial of 14 patients (Israeli et al., Liver International 2004 24; 295-307), a mixture of HB SAg+preS1+preS2 protein supplemented by the addition of liver extracted proteins was used. Due to the smaller size of the trial, only 4 of the 14 patients were HBeAg positive and consequently no patients were seen to seroconvert (the corresponding rate in the previous trial with 19 HBeAg patients would have predicted only 1 out of 4 at most to seroconvert from HBeAg positive to HBeAg negative). A rebalancing of the Th2 response compared to the Th1 response was also observed in this trial. Prior to treatment, 6 of the patients had elevated levels of IL-10. All 6 reverted to lower levels after treatment, and 5 out of 14 patients showed an increase in IFN-gamma secreting cells. Similar to the earlier study, in this clinical trial, 6 out of 10 patients showed an increase in antigen-specific T cell responses after treatment.

One embodiment of the invention provides a method for preventing or treating Hepatitis B infection, such as chronic Hepatitis B infection, Hepatitis B-associated liver disease and/or Hepatitis B-associated cancer such as hepatocellular carcinoma (HCC), in a non-human mammal and human patients, which method includes:

co-administering to the non-human mammal or human patient, for example, via oral administration:
(i) one or more HepB antigens such as one or more preselected HepB antigens, for example HepB proteins or peptides that are recombinantly or synthetically manufactured; and
(ii) one or more of: heat-treated blood (HTB) or a heat-treated RBC blood fraction, such as autologous or heterologous (from the same species of mammal or a different species of mammal), a cell membrane fraction of the foregoing, a protein extract of any of the foregoing, alpha-synuclein protein, such as mammalian alpha-synuclein protein, such as human alpha-synuclein protein, or fragments thereof of any of said alpha-synucleins. The utilized components of (i) and (ii) may be mixed together or provided as mixed and administered as one composition or may be co-administered as separate compositions.

The mammal or human may, for example, be currently infected with Hepatitis B virus, such as chronically infected with the virus, or was previously but not currently infected with Hepatitis B virus. Treatment of a chronic or active HepB infection can result in conversion to an inactive carrier state. In subjects with HCC, treatment can shift the immune response toward Th1 and prevent/delay progression of the HCC. In HBV infected subjects, treatment can prevent or delay the progression of HBV-associated liver diseases and progression to cancers such as HCC.

A related embodiment of the invention provides a pharmaceutical composition, such as an oral pharmaceutical composition for preventing or treating Hepatitis B infection, such as chronic Hepatitis B infection, Hepatitis B-associated liver disease and/or Hepatitis B-associated cancer such as hepatocellular carcinoma (HCC), in a non-human mammal and human patients, which composition includes:
(i) one or more HepB antigens such as one or more preselected HepB antigens, for example HepB proteins or peptides that are recombinantly or synthetically manufactured; and
(ii) one or more of: heat-treated blood (HTB) or a heat-treated RBC blood fraction, such as autologous or heterologous (from the same species of mammal or a different species of mammal), a cell membrane fraction of the foregoing, a protein extract of any of the foregoing, alpha-synuclein protein, such as mammalian alpha-synuclein protein, such as human alpha-synuclein protein, or fragments thereof of any of said alpha-synucleins.

The one or more Hepatitis B antigens in the preceding embodiments may, for example, be or include one or more (in any combination) of HBsAg (surface antigen, S-protein) such as SEQ ID NO: 37 (adw serotype) and/or SEQ ID NO: 38 (adr serotype), HB pre-S1 protein (SEQ ID NO: 39), HB pre-S2 protein (SEQ ID NO: 40), HBeAg (HepB envelope antigen; e.g., SEQ ID NO: 41), and HBcAg (HepB core antigen; e.g. SEQ ID NO: 42); recombinant forms of each are well known and commercially available. Alpha-synuclein in these embodiments may, for example, be recombinant (such as recombinant human alpha-synuclein, SEQ ID NO: 3) or purified from a tissue source. The Hepatitis B antigen(s) and alpha-synuclein(s) may, for example, each be provided in an at least substantially pure form for use in the embodiments.

The measurement of immune reactivity to selected antigens is a common practice in either diagnosing the presence of a disease state or delineating the stage or progression of a disease state. An example of the former is establishing whether an individual has been exposed to a particular antigen such as a viral, fungal or environmental agent. An example of the latter is the determining the status of infection in a person exposed to Mycobacterium tuberculosis (TB) where different immune reactions are characteristic of different stages. Existing assay methods for detecting antigen-specific cellular responses are disclosed, for example, in U.S. Pat. No. 5,955,077, U.S. Pat. No. 6,991,797 and U.S. Pat. No. 7,575,870, each of which is hereby incorporated by reference in its entirety. For diagnostic purposes, the enhancement of specific immune reactivity by the present invention may offer increased sensitivity where exposure to selected antigens can be detected at an earlier time point and enhanced detection of positive responses over background, thereby permitting determinations of positivity in otherwise ambiguous circumstances.

Accordingly, another embodiment of the invention provides an assay method for determining whether a sample of cells mounts an antigen-specific response to one or more preselected antigens and/or for quantifying the extent to which a sample of cells mounts an antigen-specific response to one or more preselected antigens, said method embodiment including the steps of: providing an isolated sample of cells, such as a sample of blood cells, such as whole blood, or a white blood cell fraction or PBMCs or T-cells; providing alpha-synuclein such as mammalian alpha-synuclein such as human alpha-synuclein and/or a fragment of any of the foregoing; providing at least one such as one preselected antigen, such as but not limited to an antigen which is a synthetic or recombinant peptide or protein; contacting the sample of cells with both the alpha-synuclein (any of the aforementioned varieties) and/or fragments thereof and the at least one preselected antigen; and measuring the resulting response, such as cell type-specific response, of the sample of cells or a subset of cells of interest therein, such as T-cells, for example, by quantifying the release of or increase of gene expression of or translation of one or more cytokines, such as interferon gamma, for example using conventional and commercially available means such as ELISA assays for protein quantitation or quantitative RT-PCR for quantification of gene expression. The sample of cells may, for example, be obtained from a non-human mammal or a human. The synuclein or fragments thereof and the at least one preselected antigen may, for example, be mixed with each other (or be premixed) before being contacted with, such being added to, the sample of cells. Parallel steps may be run with all of the same components except for, i.e., excluding, the at least one preselected antigen (and optionally using a neutral “dummy antigen” in its place) as a control arm with the final measurements used as a negative control for the presumptive antigen stimulation arm.

A related embodiment of the invention provides an assay composition that includes, as mixture, an isolated sample of cells obtained from a subject, such as a sample of blood cells, such as whole blood, or a white blood cell fraction or PBMCs, said cells being, for example, non-human mammalian cells or human cells; alpha-synuclein such as mammalian alpha-synuclein such as human alpha-synuclein and/or a fragment of any of the foregoing; and at least one such as one preselected antigen, such as but not limited to an antigen which is a synthetic or recombinant peptide or protein.

Another embodiment of the invention provides a vessel having an inner surface defining a volume, such as but not limited to a blood collection tube or a well of a microwell plate, wherein said inner surface is coated (i) at least in part with at least one preselected antigen, such as a protein or peptide antigen, which antigen is not alpha-synuclein protein or a fragment thereof, and (ii) at least in part with alpha-synuclein protein such as recombinant or synthetic alpha-synuclein protein or a fragment thereof. The surface part coated in (i) and (ii) may be the same or different, or partially overlap. The vessel may, for example, be dry and not filled with any liquid as when stored for use and can be at least partially filled with liquid for use. The antigen and the alpha-synuclein protein or a fragment thereof may be dried on the part(s) of the inner surface.

A further embodiment of the invention provides a composition of matter that includes:
the aforementioned vessel embodiment; and
a quantity of whole blood or a fraction thereof including T-cells (such as a PBMC fraction),

wherein the quantity of whole blood or a fraction thereof including T-cells is contained by the vessel, and

wherein the quantity of whole blood or a fraction thereof including T-cells is in contact with both the at least one preselected protein or peptide antigen and the synthetic or recombinant alpha-synuclein protein or fragment thereof in the vessel. The whole blood or fraction thereof may be fresh.

A related embodiment of the invention provides a composition of matter that includes:
a vessel having an inner surface defining a volume, such as but not limited to a blood collection tube or a well of a microwell plate;
at least one preselected antigen, such as a preselected protein or peptide antigen, which antigen is not alpha-synuclein protein or a fragment thereof, inside the vessel; and
synthetic or recombinant alpha-synuclein protein or a fragment thereof inside the vessel.
   The vessel may contain a quantity of whole blood or a fraction thereof wherein the quantity of whole blood or a fraction thereof including T-cells is in contact with both the at least one preselected antigen and the synthetic or recombinant alpha-synuclein protein or fragment thereof in the vessel. The whole blood or fraction thereof may be fresh.

A further embodiment of the invention provides a cytokine release assay method, such as an interferon-gamma release assay (IGRA) method that includes the steps of:
providing a vessel embodiment as described herein;
adding a quantity of whole blood or a fraction thereof including T-cells, which may be fresh, to the vessel whereby T-cells in the quantity of whole blood or the fraction thereof including T-cells are contacted with the at least one preselected antigen and the alpha-synuclein protein or fragment thereof; and
measuring the quantity of a cytokine, such as interferon-gamma, released by T-cells in the quantity of whole blood or fraction thereof in response to said adding step.
   The method may also include an active mixing step just after the adding step and before the measuring step. The method may further include after the adding step and before the measuring step, a step of incubating the vessel at a temperature permissive for viability of T-cells such as 37° C. for a period of time such as 8-26 hours or any subrange or value therein. An incubation time that is not sufficient to effect differentiation of precursor effector T-cells to immediate effector T-cells may be used. If an active mixing step is included, the incubation step may be performed after the mixing step and before the measuring step

A related embodiment of the invention provides a cytokine release assay method, such as an interferon-gamma release assay (IGRA) method, for quantifying T-cell responsiveness to at least one preselected antigen that includes the steps of:
forming a mixture including:
   an ex vivo quantity of whole blood or a fraction thereof including T-cells,
   a quantity of at least one preselected antigen, such as a protein or peptide antigen, that is not alpha-synuclein protein or a fragment thereof, and
   a quantity of alpha-synuclein protein or a fragment thereof;
measuring the quantity of a cytokine, such as interferon-gamma, released by T-cells in the quantity of whole blood or fraction thereof in response to contact with the at least one preselected antigen and the alpha-synuclein protein.
   The method may further include after the mixture-forming step and before the measuring step, a step of incubating the vessel at a temperature permissive for viability of T-cells such as 37° C. for a period of time such as 8-26 hours or any subrange or value therein. An incubation time that is not sufficient to effect differentiation of precursor effector T-cells to immediate effector T-cells may be used.

A further embodiment of the invention provides a method of manufacturing a vessel, such as one or more wells of a microwell plate or a blood collection tube, for use in an antigen specific T-cell response assay, such as a cytokine release assay, such as an interferon-gamma release assay, that includes the steps of:
providing a vessel having an inside surface defining a volume, such as a blood collection tube or a well of a microwell plate;
providing a liquid composition, such as an aqueous solution or suspension, including at least one preselected antigen, such as a protein or peptide antigen;
providing a liquid composition, such as an aqueous solution or suspension, including alpha-synuclein or a fragment thereof,
wherein the liquid composition including the at least one preselected antigen and the liquid composition including alpha-synuclein or a fragment thereof may be the same liquid composition or different liquid compositions;
contacting the inner surface of the vessel with the liquid composition(s) including the at least one antigen and the alpha-synuclein or fragment thereof, for example, by at least partially filling the volume of the vessel with composition(s); and
drying the vessel.
   Optionally, before the drying step, the vessel volume may be at least substantially emptied of the composition by pouring out (gravity), with or without agitation/tapping, and/or aspiration.

Blood collection tubes and microwell plates as recited in the various embodiments may, for example, be composed of glass or synthetic polymer such as polyethylene or polypropylene, as known in the art. Any of the cytokine release assay method embodiments above may, for example, instead of or in addition to measuring cytokine release, measure mRNA expression of T-cell specific mRNA such as a cytokine, such as interferon-gamma. In any of embodiments involving alpha-synuclein or a fragment thereof, as an alternative or additional antigen-specific response enhancer to the alpha-synuclein or fragment thereof, an HLA protein or a fragment thereof as described herein may be used, added, and/or included. In any of the embodiments, the antigen(s) may also exclude HLA protein and/or fragments thereof.

In the aforementioned vessel, assay method, assay composition embodiments, the at least one preselected antigen may, for example, be or include an antigen of or associated with a pathogenic cellular organism such as a bacteria, fungi, protozoan, amoeba or a virus. Antigen-specific reactivity detected from the sample of cells by the measuring step is indicative or strongly predictive that the subject from which the sample was obtained is currently infected with the pathogen. In this manner, a diagnosis can be provided. The at least one preselected antigen may, for example, be or include one or at least one protein/peptide antigen of Mycobacterium tuberculosis bacterium, for example, ESAT-6, or a peptide fragment of said antigen, for example, one or more fragments of ESAT-6, and may, for example, be or include one or more synthetic peptides or recombinant proteins. For example, full-length ESAT-6 protein may be used (such as SEQ ID NO: 17 herein), and/or any of the Mycobacterium tuberculosis ESAT-6 derived peptide antigens disclosed in U.S. Pat. No. 7,632,646 (such as SEQ ID NOS: 18-25 herein) may be used, and/or any of the non-ESAT-6 antigens in Mustafa et al., Clinical Infectious Diseases 2000; 30(Suppl 3): S201-5 (such as SEQ ID NOS: 26-36 herein) may be used, each alone or in any combination.

Alpha-synuclein protein or fragments thereof may, for example, be added to the Mycobacterium tuberculosis (TB) challenge peptides used in commercially available interferon-gamma release assays (IGRAs) for detecting M. tuberculosis infection, such as the FDA-approved tests QuantiFERON-TB Gold In-Tube (QFT-GIT) (Cellestis/Qiagen, Venlo, Limburg) and T-SPOT.TB (Oxford Immunotec, Abingdon, UK), to enhance their performance. The alpha-synuclein or fragment(s) thereof may, for example, be added to a concentration of 1-40 μg/ml, or any subrange or value therein, such as 10-30 μg/ml, in the incubation mixture (blood/cells plus antigens and alpha-synuclein or fragment(s) thereof) of the assays.

The antigens used in QFT-GIT and T-SPOT.TB are selected from the RD1 portion of the TB genome, which is absent from BCG vaccine strains and most commonly occurring non-tuberculosis mycobacteria (NTM). Both QFT-GIT and T-SPOT.TB use ESAT-6 and CFP-10 peptide while QFT-GIT also includes TB 7.7 antigen. Both IGRAs include internal controls, termed the nil and mitogen, in addition to the stimulatory TB peptide antigen. The nil determines the amount of interferon gamma detected after incubation without antigens. The result from the nil control is subtracted from the result after stimulation with the TB antigens to determine the interferon gamma that is attributable to TB. The mitogen control is used to confirm that a test subject's cells are capable of responding to antigen stimulation and that the test was performed correctly. Phytohemagglutinin (PHA) is used as a nonspecific antigen stimulant and failure to respond appropriately suggests an inadequate number of functional effector T-cells or an error in processing the blood or performing the test.

QFT-GIT and T-SPOT.TB measure interferon-gamma differently. QFT-GIT employs ELISA that measures interferon-gamma produced in heparinized whole blood after stimulation. Blood is collected into 3 specialized tubes, approximately 1 ml in each of the nil, TB antigen, and mitogen tubes. The tubes are shaken after collection to ensure the antigens dried on the inner surface of the TB and mitogen tubes are adequately mixed with the blood. Tubes are then incubated for 16-24 hours at 37° C. After incubation, the tubes are centrifuged, plasma is extracted, and interferon-gamma levels are measured by ELISA.

T-SPOT.TB uses an enzyme-linked immunospot (ELISPOT) method that determines the quantity of effector T cells responding to antigen stimulation. For most test subjects, 8 mL of heparinized whole blood is adequate to supply enough cells. From the blood, peripheral blood mononuclear cells (PBMCs) are separated, washed, and counted. The PBMCs are then added into microtiter wells at a concentration of 250,000±50,000 PBMCs per well. Each test employs 4 wells: a nil control, a mitogen-containing PHA, and 2 separate wells for ESAT-6 and CFP-10. The microtiter plates are then incubated at 37° C. with 5% CO₂ for 16-20 hours. Released interferon-gamma is captured by specific antibodies on the base of the wells and quantified by a colorimetric enzyme-linked immunoassay.

In any of the assay method embodiments of the invention, the alpha-synuclein or fragment(s) thereof may, for example, be present/added to a concentration of 1-40 μg/ml, or any subrange or value therein, such as 10-30 μg/ml, in the incubation mixture (blood/cells plus antigens and alpha-synuclein or fragment(s) thereof) of the assays. The preselected antigens may also, for example, be present/added to a concentration of 1-40 μg/ml, or any subrange or value therein, such as 10-30 μg/ml, in the incubation mixture (blood/cells plus antigens and alpha-synuclein or fragment(s) thereof) of the assays. Vessel embodiments of the invention, such as blood collection tube and microwell embodiments may include/contain a sufficient amount of alpha-synuclein or fragment(s) thereof and preselected antigen(s) to obtain the aforementioned concentrations when a recommended assay volume of blood or cells is added to and contained by the vessel. For example, for an assay using 1 ml of fresh blood, a vessel embodiment of the invention having an internal volume of at least 1 ml may contain, such as be internally coated with, 20 μg recombinant human alpha-synuclein protein and 5-10 μg total of one or more preselected synthetic peptide antigens, such as but not limited to one or more M. tuberculosis ESAT-6 and/or CFP-10 peptide antigens.

In embodiments that include/recite cells, at least one preselected antigen and alpha-synuclein protein (or a fragment thereof) and/or HLA protein (or a fragment thereof), it should be understood that the recited at least one preselected antigen refers to one or more antigens that are, at least substantially, not provided by the cell sample itself, i.e., are exogenous with respect to the cells (and added to it), versus various possible antigens that may be endogenous to and already present in the obtained cell sample. Similarly, in such embodiments, the recited alpha-synuclein protein (or a fragment thereof) and/or HLA protein (or a fragment thereof) refers to molecules that are exogenous to the cell sample (and added to it), versus those that may be endogenous to and already present in the obtained cell sample (for example, a fresh blood sample may include a small amount of endogenous alpha-synuclein protein and/or HLA protein). Thus, for example, in the aforementioned assay method embodiments, the recited quantity of at least one preselected antigen and quantity of alpha-synuclein protein or fragment thereof recited in the assay method refer to things admixed with the quantity of blood or fraction thereof, not things provided by the quantity of blood or fraction thereof itself.

The proteins used in various embodiments of the invention, such as HLA proteins or fragments thereof and alpha-synuclein protein or fragments thereof may, for example, be recombinant or may be purified from biological tissue sources, such as blood. The proteins may be at least substantially purified and/or at least substantially pure. By “at least substantially purified” and “at least substantially pure” it is intended is that the recited composition(s) need not be perfectly purified or perfectly pure. A fragment of a protein may, for example, include at least 5, such as at least 10, consecutive amino acids of the amino sequence of the protein but less than the full length sequence of the protein. A fragment of a protein may, for example, comprise consecutive amino sequence of the protein which is less than the full length of the protein, for example, 10-99% of the full length of the protein or any subrange of percentages therein, such as 10-90%, or any percent figures therein that correspond to any of the non-full length subsequences (of consecutive amino acids) of the protein. Peptide synthesis, as known in the art, may also be used to provide smaller fragments of larger proteins or small proteins.

Synthetic peptides used in the embodiments of the invention may, for example, be in the range of 5-150 amino acids long, such as 5-40 amino acids long, or any subrange therein or any number of amino acids within said ranges. For example, the synthetic peptides may be 10-30 amino acids long, 10-25 amino acids long, 10-20 amino acids long or 10-16 amino acids long.

As used herein, the term antigen means a molecule that presents one or more immune epitopes. These epitopes may be recognized by immune cells such as T-cells. Such epitopes, and thus the antigens themselves, may be immune reactivity-promoting (immunostimulatory; pro-effector T-cell) or immune suppression-promoting (immunosuppressive; pro-regulatory T-cell). The assay embodiments of the invention may be used to measure release of cytokines associated with antigen-specific effector T-cell responses, such as interferon-gamma (IFN-gamma), interleukin-6 (IL-6) and interleukin-8 (IL-8), as well as release of cytokines associated with antigen-specific regulatory/suppressive T-cell responses, such as interleukin-10 (IL-10).

The immunogen may, for example, be a vaccine immunogen. The vaccine immunogen may, for example, be an infectious disease immunogen or a tumor/cancer antigen vaccine immunogen. The infectious disease immunogen may, for example, be a vaccine immunogen against a cellular or viral pathogen, may for example be a live or killed/inactivated form of the pathogen or a derivative/extract thereof, and/or may for example include or consist of one or more purified antigens such as synthetic antigen molecules for the pathogen, such as synthetic peptides or recombinant proteins. The tumor/cancer vaccine immunogen may, for example, include or consist of cancer cells, parts of cancer cells, or pure tumor/cancer antigens isolated from the cells or produced synthetically, such as, without limitation, synthetic peptides or recombinant proteins.

Each of the patents and other publications cited in this disclosure is hereby incorporated by reference in its entirety.

Although the foregoing description is directed to preferred embodiments of the invention, it is noted that other variations and modifications will be apparent to those skilled in the art, and may be made without departing from the spirit or scope of the invention. Wherever in this disclosure the terms include(s)/including or comprise(s)/comprising have been used, it should be understood that corresponding embodiments and disclosures reciting consist(s)/consisting and consist(s)/consisting essentially of are also taught. Moreover, features described in connection with one embodiment of the invention may be used in conjunction or combination with other embodiments, even if not explicitly exemplified in conjunction or combination in this disclosure.

1-25. (canceled)

26. In a cytokine release assay method comprising contacting T-cells ex viva with at least one preselected antigen that is not alpha-synuclein and measuring the quantity of a cytokine released by the T-cells in response to said contacting, the improvement comprising contacting the T-cells with both the at least one preselected antigen and alpha-synuclein.

27. The cytokine release assay method of claim 26, wherein the method is an interferon-gamma release assay (IGRA) method and the cytokine is interferon-gamma.

28. The cytokine release assay method of claim 26, wherein the alpha-synuclein is human alpha-synuclein.

29. The cytokine release assay method of claim 26, wherein the alpha-synuclein is recombinant alpha-synuclein.

30. The cytokine release assay method of claim 29, wherein the recombinant alpha-synuclein is recombinant human alpha-synuclein.

31. A cytokine release assay method, comprising the steps of:
providing a vessel having an inner surface defining a volume, wherein said inner surface is coated (i) at least in part with at least one preselected protein or peptide antigen that is not alpha-synuclein protein or a fragment thereof, and (ii) at least in part with recombinant alpha-synuclein protein or a fragment thereof;
adding a quantity of whole blood or a fraction thereof comprising T-cells to the vessel whereby T-cells in the quantity of whole blood or the fraction thereof comprising T-cells are contacted with the at least one preselected protein or peptide antigen and the alpha-synuclein protein or fragment thereof; and
measuring the quantity of a cytokine released by T-cells in the quantity of whole blood or fraction thereof in response to said adding step.

32. The cytokine release assay method of claim 31, wherein the method is an interferon-gamma release assay (IGRA) method and the cytokine is interferon-gamma.

33. The method of claim 31, further comprising the step of:
between the adding step and the measuring step, incubating the vessel for a period of time.

34. The cytokine release assay method of claim 33, wherein the method is an interferon-gamma release assay (IGRA) method and the cytokine is interferon-gamma.

35. A cytokine release assay method for quantifying T-cell responsiveness to at least one preselected antigen, comprising the steps of:
forming a mixture comprising:
an ex vivo quantity of whole blood or a fraction thereof comprising T-cells,
a quantity of at least one preselected antigen that is not alpha-synuclein protein or a fragment thereof, and
a quantity of recombinant alpha-synuclein protein or a recombinant fragment thereof;
measuring the quantity of a cytokine released by T-cells in the quantity of whole blood or fraction thereof in response to contact with the at least one preselected antigen and the alpha-synuclein protein.

36. The cytokine release assay method of claim 35, wherein the at least one preselected antigen comprises one or more of a purified protein, a recombinant protein, and a synthetic peptide.

37. The cytokine release assay method of claim 35, wherein the method is an interferon-gamma release assay (IGRA) method and the cytokine is interferon-gamma.

38. The cytokine release assay method of claim 37, wherein the at least one preselected antigen comprises one or more of a purified protein, a recombinant protein, and a synthetic peptide.

39. The method of claim 35, further comprising the step of:
between the adding step and the measuring step, incubating the vessel for a period of time.

40. The cytokine release assay method of claim 39, wherein the method is an interferon-gamma release assay (IGRA) method and the cytokine is interferon-gamma.


SEQUENCE LISTING (42 sequences)

SEQ ID NO: 1 - protein, 284 aa, Homo sapiens
GSHSMRYFYTAMSRPGRGEPRFISVGYVDDTQFVRFDSDAASPREEPRAPWIEQEGPEYWDRNTQICKTNTQTYRESLRNLRGYYNQSEAGSHTLQRMYGCDVGPDGRLLRGHDQYAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPI

SEQ ID NO: 2 - protein, 313 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSS

SEQ ID NO: 3 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDVDVIGLTFRRDLYFSRVQVYPPVGAASTPTKLQESLLKKLGGNTYPFLLTFPDYLPCSVMLQPAPQDSGKSCGVDFEVKAFATDSTDAEEDKIPKKSSVRYLIRSVQHAPLEMGPQPRAEATWQFFMSDKPLHLAVSLNREIYFHGEPIPVTVDVTNNTEKTVKKIKACVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESIQDANLVFEEFARHNLKDAGEAEEGKRDKNDADE

SEQ ID NO: 5 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 6 - protein, 20 aa, Artificial Sequence
  Synthetic fragment of human S-antigen
GEPIPVTVDVTNNTEKTVKK

SEQ ID NO: 7 - protein, 15 aa, Artificial Sequence
  Synthetic fragment of human S-antigen
VTVDVTNNTEKTVKK

SEQ ID NO: 8 - protein, 14 aa, Artificial Sequence
  Synthetic HLA peptide B27PD
ALNEDLSSWTAADT

SEQ ID NO: 9 - protein, 15 aa, Artificial Sequence
  Synthetic fragment of human S-antigen
IFKKISRDKSVTIYL

SEQ ID NO: 10 - protein, 15 aa, Artificial Sequence
  Synthetic fragment of human S-antigen
VKGKKVYVTLTCAFR

SEQ ID NO: 11 - protein, 15 aa, Artificial Sequence
  Synthetic fragment of human S-antigen
YGQEDVDVIGLTFRR

SEQ ID NO: 12 - protein, 15 aa, Artificial Sequence
  Synthetic fragment of human S-antigen
LPLLANNRERRGIAL

SEQ ID NO: 13 - protein, 15 aa, Artificial Sequence
  Synthetic fragment of human S-antigen
DTNLASSTIIKEGID

SEQ ID NO: 14 - protein, 14 aa, Artificial Sequence
  Synthetic peptide
ALNEDLSSWTAADT

SEQ ID NO: 15 - protein, 203 aa, Homo sapiens [sequence omitted]

SEQ ID NO: 16 - protein, 1487 aa, Homo sapiens
MIRLGAPQSLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 17 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 18 - protein, 20 aa, Mycobacterium tuberculosis
MTEQQWNFAGIEAAASAIQG

SEQ ID NO: 19 - protein, 24 aa, Mycobacterium tuberculosis
EAAASAIQGNVTSIHSLLDEGKQS

SEQ ID NO: 20 - protein, 24 aa, Mycobacterium tuberculosis
VTSIHSLLDEGKQSLTKLAAAWGG

SEQ ID NO: 21 - protein, 24 aa, Mycobacterium tuberculosis
GKQSLTKLAAAWGGSGSEAYQGVQ

SEQ ID NO: 22 - protein, 24 aa, Mycobacterium tuberculosis
AWGGSGSEAYQGVQQKWDATATEL

SEQ ID NO: 23 - protein, 24 aa, Mycobacterium tuberculosis
QGVQQKWDATATELNNALQNLART

SEQ ID NO: 24 - protein, 24 aa, Mycobacterium tuberculosis
ATELNNALQNLARTISEAGQAMAS

SEQ ID NO: 25 - protein, 24 aa, Mycobacterium tuberculosis
LARTISEAGQAMASTEGNVTGMFA

SEQ ID NO: 26 - protein, 15 aa, Mycobacterium tuberculosis
MTEQQWNFAGIEAAA

SEQ ID NO: 27 - protein, 15 aa, Mycobacterium tuberculosis
SAIQGNVTSIHSLLD

SEQ ID NO: 28 - protein, 15 aa, Mycobacterium tuberculosis
QKWDATATELNNALQ

SEQ ID NO: 29 - protein, 15 aa, Mycobacterium tuberculosis
NNALQNLARTISEAG

SEQ ID NO: 30 - protein, 15 aa, Mycobacterium tuberculosis
NLARTISEAGQAMAS

SEQ ID NO: 31 - protein, 15 aa, Mycobacterium tuberculosis
WNFAGIEAAASAIQG

SEQ ID NO: 32 - protein, 15 aa, Mycobacterium tuberculosis
EGKQSLTKLAAAWGG

SEQ ID NO: 33 - protein, 15 aa, Mycobacterium tuberculosis
YQGVQQKWDATATEL

SEQ ID NO: 34 - protein, 15 aa, Mycobacterium tuberculosis
NVTSIHSLLDEGKQS

SEQ ID NO: 35 - protein, 15 aa, Mycobacterium tuberculosis
IEAAASAIQGNVTSI

SEQ ID NO: 36 - protein, 15 aa, Mycobacterium tuberculosis
TATELNNALQNLART

SEQ ID NO: 37 - protein, 226 aa, Hepatitis B virus
MENITSGLLGPLLVLQAGFFLLTRILTIPQSLDSWWTSLSFLGEAPVCLGQNSQSPTRNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMIWYWGPSLYSIVCPFTPLLQIFCCLWVFI

SEQ ID NO: 38 - protein, 226 aa, Hepatitis B virus
MESTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFAGLSPTVWLSVIWMMWYWGPSLYNILSPFLPLLPIFFCLWVYI

SEQ ID NO: 39 - protein, 119 aa, Hepatitis B virus
MGGWSSKPRQGMGTNLSVPNPLGFFPDHQLDPAFGANSNNPDWDFNPNKDHWPEAHQVGAGAFGPGFTPPHGGLLGWSPQAQGILTTVPVAPPPASTNRQSGRQPTPISPPLRDSHPQA

SEQ ID NO: 40 - protein, 55 aa, Hepatitis B virus
MQWNSTTFHQALLDPKVRGLYFPAGGSSSGTVNPVPTTASPISSIFSRTGDPAPN

SEQ ID NO: 41 - protein, 195 aa, Hepatitis B virus
SKLCLGWLWGMDIDPYKEFGATVELLSFLPSDFFPSVRDLLDTASALYREALESPEHCSPHHTALRQAILCWGELMTLATWVGNNLEDPASRDLVVNYVNTNVGLKIRQLLWFHISCLTFGRETVLEYLVSFGVWIRTPPAYRPPNAPILSTLPETTVVRRRDRGRSPRRRTPSPRRRRSPSPRRRRSQSRESQC

SEQ ID NO: 42 - protein, 183 aa, Hepatitis B virus
MDIDPYKEFGASVELLSFLPSDFFPSIRDLLDTASALYREALESPEHCSPHHTALRQAILCWGELMNLATWVGSNLEDPASRELVVSYVNVNMGLKFRQLLWFHVSCLTFGRETVLEYLVSFGVWIRTPPAYRPPNAPILSTLPETTVVRRRGRSPRRRTPSPRRRRSQSPRRRRSQSRESQC